(19) United States
(12) Patent Application Publication
(10) Pub. No.: US 2023/0406906 A1
(43) Pub. Date: Dec. 21, 2023

(54) SERPIN PEPTIDES AND METHODS OF USING THE SAME

(71) Applicant: SERPIN PHARMA, LLC, Manassas, VA (US)

(72) Inventors: Dana AUSTIN, Manassas, VA (US); Cohava GELBER, Manassas, VA (US)

(21) Appl. No.: 18/148,942

(22) Filed: Jul. 13, 2023

Related U.S. Application Data
(60) Provisional application No. 63/266,444, filed on Jan. 5, 2022.

(51) Int. Cl. C07K 14/81; A61P 29/00

(52) U.S. Cl. CPC C07K 14/8121; A61P 29/00

(57) ABSTRACT

Disclosed herein are SERPIN peptides, and analogues and derivatives thereof, and uses of the same for treating various conditions associated with LRP1 or TSLP.

### CROSS-REFERENCE TO RELATED APPLICATION

The present application claims the benefit of, and priority to, U.S. Provisional Patent Application No. 63/266,444 filed Jan. 5, 2022, the disclosure of which is herein incorporated by reference in its entirety.

### SEQUENCE LISTING

This application contains an ST.26 compliant sequence listing, which is being submitted concurrently herewith in .xml format via Patent Center and is hereby incorporated by reference in its entirety. The .xml copy, created on Jan. 23, 2023, is named Serpin Pharma 138536-8001WOO1 Replacement Sequence Listing.xml and is 44,479 bytes in size.

### BACKGROUND

Serine protease inhibitors (SERPINs) are a large family of proteins that are involved in diverse biological functions such as fibrinolysis, blood coagulation, and inflammation. When SERPINs bind to their target serine proteases to inactivate the enzymatic activity, a conformational change occurs exposing a unique short peptide motif (5-11 amino acids).^{8,43} The protease-inhibitor complex binds to low-density lipoprotein receptor-related protein (LRP1) at the newly exposed short peptide motif, a process which is conserved across the entire spectrum of SERPINs such as alpha-1 antitrypsin (AAT) and antithrombin III (ATIII) (May 2013).^{22,25,43} Therefore, there is a need to develop novel SERPIN peptides and explore their prophylactic and therapeutic effects in various conditions and diseases.

### SUMMARY

In some embodiments, this technology relates to a method of reducing inflammation, including, but not limited to, innate immunity, adaptive immunity, eosinophilic inflammation, allergy, rhinitis, asthma, dermatitis, esophageal eosinophilia, eosinophilic asthma, atopic dermatitis, nasal polyps, pruritis, chronic spontaneous urticaria, and the use of a SERPIN peptide in reducing inflammation in a subject having a disease or condition associated with LRP1 or TSLP, including administering a SERPIN peptide selected from the group consisting of VKFNKPFVFL(NIe)IEQNTK (SEQ ID NO: 35), VKFNKPFVFLMIEQNTK (SEQ ID NO: 2), VKFNKPFVFLM (SEQ ID NO: 25), LRFNRPFLVVI (SEQ ID NO: 29), VRFNRPFLMII (SEQ ID NO: 31), VKFNKPFVFL(NIe) (SEQ ID NO: 40), RFNRPFLVVIR (SEQ ID NO: 41), RFNRPFLMIIR (SEQ ID NO: 42), RFNKPFVFL(NIe)R (SEQ ID NO: 43), RRRFLVVIRRR (SEQ ID NO: 44), RRRFLMIIRRR (SEQ ID NO: 45), RRRFVFL(NIe)RRR (SEQ ID NO: 46), FVFLM (SEQ ID NO: 3), and FVFL(NIe) (SEQ ID NO: 10) to the subject to reduce inflammation associated with the disease or condition associated with LRP1 or TSLP. In some aspects, the amino acid sequence of the SERPIN peptide includes the sequence of SEQ ID NO: 35 or SEQ ID NO: 2. In some aspects, the N-terminus of the SERPIN peptide is acetylated. In some aspects, the C-terminus of the SERPIN peptide is amidated. In some aspects, the SERPIN peptide is fused to one or more other peptides to form a fusion peptide or fusion protein. In some aspects, the other peptide is different from the SERPIN peptide. In aspects, the fusion peptide or fusion protein includes the SERPIN peptide and an epitope tag, a half-life extender, or both the epitope tag and the half-life extender. In some aspects, the SERPIN peptide is administered at a dose of between 0.001 mg/kg and 5 mg/kg. In some aspects, the subject is a human, and the SERPIN peptide is administered by oral administration, parenteral administration, intradermal administration, transdermal administration, topical administration, or intranasal administration. In some aspects, the SERPIN peptide is administered as a single dose.

In some aspects, disease or condition is caused by A. alternata. In some aspects, the disease or condition is rhinitis, asthma, dermatitis, or esophageal eosinophilia. In some aspects, the disease or condition is acute or neuropathic pain, nociceptive pain, or inflammatory pain. In some aspects, the disease or condition is an eosinophilic driven disease (EDD), for example, eosinophilic esophagitis (EoE), eosinophilic asthma, atopic dermatitis, nasal polyps, or chronic spontaneous urticaria. In some aspects, the disease or condition is atopic dermatitis or pruritis, and the SERPIN peptide may be administered by topical administration. In some aspects, the disease or condition is an allergic reaction, allergic inflammation, or eosinophilic driven allergic disease.

In other embodiments, this technology relates to a method of treating or the use of a SERPIN peptide in treating a subject having a disease or condition associated with LRP1 or TSLP, including administering a SERPIN peptide selected from the group consisting of VKFNKPFVFL(NIe)IEQNTK (SEQ ID NO: 35), VKFNKPFVFLM (SEQ ID NO: 25), LRFNRPFLVVI (SEQ ID NO: 29), VRFNRPFLMII (SEQ ID NO: 31), VKFNKPFVFL(NIe) (SEQ ID NO: 40), RFNRPFLVVIR (SEQ ID NO: 41), RFNRPFLMIIR (SEQ ID NO: 42), RFNKPFVFL(NIe)R (SEQ ID NO: 43), RRRFLVVIRRR (SEQ ID NO: 44), RRRFLMIIRRR (SEQ ID NO: 45), RRRFVFL(NIe)RRR (SEQ ID NO: 46), FVFLM (SEQ ID NO: 3), and FVFL(NIe) (SEQ ID NO: 10) to the subject to treat the disease or condition associated with LRP1 or TSLP, where the disease or condition is acute or neuropathic pain, nociceptive pain, or inflammatory pain. In aspects, the SERPIN peptide includes the sequence of SEQ ID NO: 35. In aspects, the N-terminus of the SERPIN peptide is acetylated. In aspects, the C-terminus of the SERPIN peptide is amidated. In aspects, the SERPIN peptide is fused to one or more other peptides to form a fusion peptide or fusion protein. In aspects, the other peptide is different from the SERPIN peptide. In aspects, the fusion peptide or fusion protein includes the SERPIN peptide and an epitope tag, a half-life extender, or both the epitope tag and the half-life extender.

In aspects, the subject is a human, and the SERPIN peptide may be administered by oral administration, parenteral administration, intradermal administration, transdermal administration, topical administration, or intranasal administration. In aspects, the SERPIN peptide is administered to the subject at a dose of between 0.001 mg/kg and 5 mg/kg, and the SERPIN peptide may be administered as a single dose. In aspects, administration of the SERPIN peptide results in reduced pain, and/or the administration prevents or reduces the development of pain.

In some embodiments, this technology relates to a method of treating or the use of a SERPIN peptide in treating a subject having a disease or condition associated with LRP1 or TSLP, including administering a SERPIN peptide comprising an amino acid sequence selected from the group consisting of VKFNKPFVFL(NIe)IEQNTK (SEQ ID NO: 35), VKFNKPFVFLM (SEQ ID NO: 25), LRFNRPFLVVI (SEQ ID NO: 29), VRFNRPFLMII (SEQ ID NO: 31), VKFNKPFVFL(NIe) (SEQ ID NO: 40), RFNRPFLVVIR (SEQ ID NO: 41), RFNRPFLMIIR (SEQ ID NO: 42), RFNKPFVFL(NIe)R (SEQ ID NO: 43), RRRFLVVIRRR (SEQ ID NO: 44), RRRFLMIIRRR (SEQ ID NO: 45), RRRFVFL(NIe)RRR (SEQ ID NO: 46), FVFLM (SEQ ID NO: 3), and FVFL(NIe) (SEQ ID NO: 10) to the subject to treat the disease or condition associated with LRP1 or TSLP, where the disease or condition is caused by A. alternata. In some aspects, the amino acid sequence of the SERPIN peptide comprises the sequence of SEQ ID NO: 35, and the N-terminus of the SERPIN peptide may be acetylated and/or the C-terminus of the SERPIN peptide may be amidated. In some aspects, the SERPIN peptide is fused to one or more other peptides to form a fusion peptide or fusion protein, and the other peptide may be different from the SERPIN peptide. In some aspects, the fusion peptide or fusion protein includes the SERPIN peptide and an epitope tag, a half-life extender, or both the epitope tag and the half-life extender.

In some aspects, the SERPIN peptide is administered at a dose of between 0.001 mg/kg and 5 mg/kg, and the subject may be a human. In some aspects, the administration is by oral administration, parenteral administration, intradermal administration, transdermal administration, topical administration, or intranasal administration, and the SERPIN peptide may be administered as a single dose. In aspects, the disease or condition is rhinitis, asthma, dermatitis, or esophageal eosinophilia, and administrating the SERPIN peptide may reduce inflammation or eosinophilic inflammation.

### BRIEF DESCRIPTION OF THE DRAWINGS

FIG. 1 demonstrates that SERPINs contain an anti-inflammatory core motif comprising the LRP1 binding sequence. In an NFκB reporter assay stimulated with LPS, the NFκB lowering activity of core SERPIN peptides was lost when truncated too short due to instability of the truncated peptides. The activity was restored upon poly arginine residues stabilizing the peptides. However, when the LRP1 binding site was truncated, the activity could not be rescued by poly arginine flanks.

FIGS. 2A-2D show that SP163M promoted neurite length and growth associated protein-43 (GAP-43) in adult primary rat DRG neurons. FIG. 2A: Representative phase contrast images of primary adult DRG neurons cultured over time. Cultures were treated with vehicle or SP163M (100 ng ml) daily for 48, 72, and 96 h. Scale bar 500 μm. FIG. 2B: Representative image of immunofluorescence to detect βIII-Tubulin in primary cultured adult DRG neurons in control and SP163M (240 nM) treated cells after 54 h. Upper panel: scale bar 200 μm; lower panel: scale bar 50 μm. Note extensive neurite length in SP163M treated neurons. FIG. 2C: Quantification of immunofluorescence analysis in control (n=144 neurons) and SP163M (n=222 neurons) samples obtained from 11 distinct cultures. SP163M significantly increased maximum neurite length compared to controls. Mann-Whitney test, sum of ranks in vehicle and SP163M treated: 23537,43625; **p<0.01. Data are expressed as mean±SEM. FIG. 2D: Primary cultured adult DRG neurons were treated with vehicle or SP163M (240 nM) for 24 h. RT-qPCR analysis of GAP-43 mRNA levels (n=8 independent studies). SP163M significantly increased the regenerative associated gene compared to vehicle. Mann-Whitney test, sum of ranks in vehicle or SP163M: 29,76, **p<0.01.

FIGS. 3A-3F show that in PC12 cells, SP163M activated transient cell signaling in an LRP1 dependent manner. FIG. 3A: Dose dependent (0-240 nM) activation of phospho-ERK1/2 by SP163M after 10 min. FIG. 3B: Time course (0-30 min) of SP163M (240 nM) activation of phospho-ERK1/2. Last lane (far right) shows activation of phospho-ERK1/2 by a known LRP1 agonist, EI-tPA (12 nM). Equal amounts of protein lysates (20 μg) were loaded per lane. Immunoblot analysis detects phospho-ERK1/2 and total ERK1/2, as a loading control. FIG. 3C: RT-qPCR analysis of LRP1 mRNA after transfection with LRP1 siRNA for 48 h. Data are presented as mean±SEM; n=3 independent experiments. T-test, T=8.024, df=4, **p<0.05. FIG. 3D: Immunoblot of LRP1 levels in PC12 cells transfected with non-targeting controls (NTC) or siLRP1 for 48 h in PC12 cells. FIG. 3E: Representative immunoblot of phospho-ERK1/2 activated by SP16 (240 nM) overtime 48 h after transfection with NTC or siLRP1. FIG. 3F: Immunoblot showing activation of phospho-Akt and phospho-ERK1/2 with vehicle or SP163M (24 or 240 nM) for 10 min and in some wells, pretreated with RAP (150 nM) for 15 min. NGF (0.36 nM) for 10 min served as a cell signaling control. Equal amounts of protein lysates (20 μg) were loaded per lane. Total ERK served as a loading control.

FIGS. 4A-4C show that SP163M modulated both early and late phase of the formalin test. FIG. 4A: Time-course of formalin (2.5%) induced paw licking in C57BL6 mice. Vehicle or SP163M (0-2 μg/g s.c.) was given 1 h prior to formalin injection into the hind paw. FIG. 4B: Quantification of area under the curve (AUC) for vehicle and SP163M (0.02, 0.2 and 2 μg/g) during the early phase. Data are expressed as mean±SEM; One-way ANOVA, F=9.523, ***p<0.001. Tukey's post hoc test, vehicle (n=9) versus SP163M 0.02 μg/g (n=8), *p<0.05; vehicle versus SP16 0.2 μg/g (n=8), ***p<0.001; vehicle versus SP163M 2.0 μg/g (n=8), **p<0.01. FIG. 4C: Quantification of area under the curve (AUC) for vehicle and SP163M (0.02, 0.2 and 2 μg/g) during the late phase. Data are expressed as mean±SEM; One-way ANOVA, F=10.2, ****p<0.0001. Tukey's post hoc test, vehicle (n=9) versus SP163M 0.02 μg/g (n=8), p=ns; vehicle versus SP16 0.2 μg/g (n=9), ***p<0.005; vehicle versus SP163M 2.0 μg/g (n=8), ***p<0.005.

FIGS. 5A-5C show that systemically administered SP163M and EI-tPA attenuated acute nociception induced by intraplantar capsaicin. FIG. 5A: Nociceptive related behaviors (time spent licking) increased in both male and female mice after intraplantar injection of capsaicin (20 μg) when compared to vehicle (cyclodextrin: 20%). Data are expressed as mean±SEM. Kruskal-Wallis test ****p<0.0001. Dunn's multiple comparisons test post hoc test male versus female without capsaicin (n=6), p=ns; male versus female with capsaicin (n=8), p=ns; male without capsaicin versus male with capsaicin **p<0.01; female without capsaicin versus female with capsaicin *p<0.5. FIG. 5B: Nociceptive related behaviors (time spent licking) after administration of LRP1 interactors, SP163M (2 μg/g) or EI-tPA (2 μg/g) in male mice with intraplantar capsaicin over 10 min. Data are mean±SEM. One-way ANOVA, F=21.28 ***p<0.001. Tukey's post hoc test, vehicle (n=13) versus SP163M (n=8), **p<0.01; vehicle versus EI-tPA (n=6), **p<0.01; FIG. 5C: Nociceptive related behaviors (time spent licking) after administration of LRP1 interactors, SP163M (2 μg/g) or EI-tPA (2 μg/g) in female mice with intraplantar capsaicin over 10 min. Data are mean±SEM. One-way ANOVA, F=14.20 ***p<0.005. Tukey's post hoc test, vehicle (n=13) versus SP16 (n=8), ***p<0.005; vehicle versus EI-tPA (n=6), **p<0.01.

FIGS. 6A-6E show that systemically administered SP163M treatment blocked the development of mechanical hypersensitivity and inflammatory cell recruitment after PNL. FIG. 6A: Tactile allodynia developed after PNL and were sustained for 14 days. SP163M (2 μg/g) delivered daily and subcutaneously significantly prevented the development of tactile allodynia for 9 days post-injury (**p<0.01). Data are expressed as mean±SEM (n=7 mice/group). Two-way ANOVA, days (main effect) F(6,42)=2.51, *p<0.05; treatment (main effect) F(1,42)=57.91, ****p<0.0001; treatment×day (interaction) F(6,42)=0.7512 p=ns. Sidak's multiple comparison test vehicle versus SP16 post injury, day 2 **p<0.01, day 4 **p<0.01, day 7 *p<0.05, day 9 *p<0.05. FIGS. 6B and 6D: Immunoblot analysis of injured sciatic nerve two days after PNL in vehicle and SP163M treated mice. SP163M treatment reduced inflammatory cell infiltration (CD11b) and suppressed TLR4. FIG. 6C: Densitometric analysis of CD11b. One-way ANOVA, F=39.05; ****p<0.0001, Tukey's post hoc test, contra (n=5) versus vehicle (n=6) ***p<0.005, vehicle versus SP16 (n=5) **p<0.01. FIG. 6E: Densitometric analysis of TLR4. One-way ANOVA, F=18.54; ****p<0.0001, Tukey's post hoc test contra (n=6) versus vehicle (n=6), ***p<0.005 vehicle versus SP163M (n=6) ***p<0.005. All data are expressed as mean±SEM.

FIGS. 7A-7C show that inflammatory cell recruitment and satellite cell activation were reduced by SP163M after PNL. FIG. 7A: Transverse sections of L4 DRG immunostained for CD11b and GFAP after vehicle or SP163M treatment two days post PNL. Note abundant immunoreactivity (brown) identifying CD11b (black arrows, upper panels) in between neuronal cell bodies and close to blood vessels or identifying GFAP (black arrows, lower panels) surrounding the neuronal cell bodies in injured vehicle-treated (left) or SP163M-treated DRGs (right). CD11b and GFAP immunoreactivity in SP163M treated DRGs are minimal. Nuclei are stained with hematoxylin (blue). FIG. 7B: Quantification of CD11b in DRGs. Mann-Whitney test, sum of ranks in vehicle and SP163M treated: 23537,43625; **p<0.01. Data are expressed as mean±SEM. Mann Whitney U test, sum of ranks in vehicle (n=8) and SP163M (n=6): 77, 28; *p<0.05. FIG. 7C: Quantification of GFAP in DRGs. Mann Whitney U test, sum of ranks in vehicle (n=7) and SP163M (n=9): 91, 45; ***p<0.005.

FIGS. 8A-8C show that SP163M blocked IL-13 stimulated Stat6 phosphorylation. FIG. 8A: At 30 minutes post IL-13 induction, STAT6 was phosphorylated in vehicle and A1AT treated cells but not in SP163M treated cells. FIG. 8B: This reduction in STAT6 phosphorylation by SP163M persisted for several hours post-treatment (many different experimental replicates shown). FIG. 8C shows that the SP163M reduction of phosphorylated STAT6 was dependent on expression of LRP1. Using CRISPER/CAS9 technology, an LRP1 knockout esophageal EPC2 cell line was created. In the control cells (with LRP1) SP163M was capable of reducing phospho-STAT6 expression; however, in the LRP1 knockout cell line, SP163M was not capable of reducing phosphorylated STAT6.

FIGS. 9A-9B show that SP163M reduced eosinophilic esophagitis in a model of eosinophilic esophagitis using A. alternata as allergen. FIG. 9A: Representative images of anti-MBP staining of esophageal sections following allergen (A. alternata) or control (saline) challenges. FIG. 9B: Quantification of eosinophils in the esophagus; data shown are the number of eosinophils per high power field (HPF).

FIGS. 10A-10C show that SP163M inhibited TSLP and reduced cell death in human keratinocytes. FIG. 10A: HaCat cells were either left untreated or induced with Poly IC (50 μg/ml) for 24 hours. Cells were pretreated with either vehicle or SP163M (100 μg/ml). TSLP was measured in the supernatant via ELISA (p=0.0045 vs. vehicle treated Poly IC induced cells). FIG. 10B: HaCat cells treated as in FIG. 10A were analyzed via the CellTiter-Glo® Luminescent Cell Viability Assay by Promega. The percent change from untreated (no Poly IC) cells were compared between SP163M vs. vehicle treated poly IC induced control (p=0.0068). FIG. 10C: HaCat cells were insulted with TNFα and immunoblotted for phospho-IkBa (Ser32). GAPDH served as a loading control and signal normalization.

FIG. 11 shows that SP163M improved outcomes in an atopic dermatitis animal model. Dermatitis was induced on the skin of BALB/c mice with a series of calcipotriol (MC903) and OVA challenges over an 8-week period. SP163M (100 μg/mouse), A1AT (2 mg/mouse) or vehicle (DDW) was given twice weekly starting at 4 weeks. The number of eosinophils infiltrating the epithelial sections were quantified by MBP staining.

FIGS. 12A-12B show that SP16 inhibits key allergic inflammatory mediators. FIG. 12A: Poly I:C-induced TSLP production by SPINK7 knockout EPC2 cells (Human esophageal epithelial cells). SPINK7 KO cells and control cells were plated in high calcium and high density, for 48 hours before being treated with SP16 or 7G and Poly I:C (5 μg/ml, or untreated) for 8 hours. TSLP production in the supernatant was measured by ELISA. FIG. 12B: primary esophageal epithelial cells (EPC2) were treated with SP16 and then stimulated with IL-13, a TH2 response mediated cytokine, to induce CCL26. SP16 significantly reduced CCL26 release dose dependently.

FIG. 13A-13C shows that SP16 reduces inflammatory markers in the bronchoalveolar lavage fluid of mice following OVA challenge. Mice were subjected to the OVA-challenge allergic inflammation model and treated with vehicle or SP16 during the challenge phase. The bronchoalveolar lavage fluid (BALF) was analyzed for cytokines via ELISA. FIG. 13A: SP16 significantly reduces CCL24 following OVA-challenge in sensitized mice. FIG. 13B: SP16 significantly reduces IL-1a following OVA-challenge in sensitized mice. FIG. 13C: SP16 significantly reduces IL-2 following OVA-challenge in sensitized mice.

FIG. 14 shows that SP16 treatment mediates TH2 driven inflammatory cytokines in OVA model. Mice were subjected to the OVA-challenge allergic inflammation model and treated with vehicle (saline), dexamethasone (1 mg/kg) (pos cont.) or SP16 (50 μg), for 4 total treatments during the challenge phase (intranasal OVA, 4 treatments over 8 days). BALF and lung tissue was analyzed for cytokines via ELISA. Cells in the BALF were counted with hemacytometer and total WBC count shown.

FIG. 15A-15B shows that SP16 reduces eosinophilic infiltration into the lung in OVA model. FIG. 15A: mice were subjected to the OVA-challenge allergic inflammation model and treated with vehicle, SP16, or A1AT during the challenge phase. The BALF was collected and analyzed for the percent of eosinophils and compared to saline (non-challenged) mice, vehicle OVA induced mice, or SP16 treated OVA induced mice. FIG. 15B shows results of a flow cytometry analysis of the BALF from FIG. 15A.

FIG. 16A-16C shows that SP16 improves outcomes in an AD-like inflammatory skin model. SP16 (in 70% ethanol) was applied topically to the ears on the days of the MC903 applications (MC903+SP16). Drug vehicle (70% EtOH) was applied to control animals (MC903) and normal control animals (NC) received EtOH in place of MC903. In-life parameters: Dermatitis score (FIG. 16A) and ear volume (FIG. 16B) measured on days 0, 3, 7, 9, 11 and 14. As shown in FIG. 16C, pictures were taken on the last day of the study (Day 15) and a randomly chosen representative mouse from each group is shown. Clinical Dermatitis Score (score 0-4 for each parameter)—erythema, scale/dryness, edema, excoriation/erosion (total score 0-16).

FIG. 17 shows that SP16 treatment results in attenuation of pruritis through PAR2 and TSLP inhibition: Mice were induced with AD using a daily application of MC903. Mice were treated with SP16 in 70% EtOH (MC903+SP16) or drug vehicle (MC903). Normal control (NC) mice were treated with EtOH only. Ears were measured for PAR2 protein expression through western blot analysis (quantitative analysis after normalization to beta-actin shown) (n=3-5). TSLP was measured in both the ears and serum by ELISA (N=3-5). Scratching was assessed over a 3 minute period after a 5-minute acclimatization period and the number of scratches are shown (n=3). IL-4 was measured in ear lysate via ELISA (N=3-5).

### DETAILED DESCRIPTION

Disclosed herein are C-terminal peptides derived from the SERPIN molecule and variants and derivatives thereof, as well as their uses in prevention or treatment of various conditions by targeting LRP1. In certain embodiments, the SERPIN peptides are isolated peptides. In certain embodiments, the SERPIN peptides are synthetic peptides.

Also disclosed herein are pharmaceutical compositions comprising the SERPIN-derived peptides and methods of using the same to treat a number of conditions where a dysregulated immune response or impaired endocytic function, or diseases in which LRP1 mediation could contribute to pathology, such as in conditions associated with peripheral nerve injury (and resulting pain), and allergic inflammation. The unexpected regenerative and healing properties of these peptides allow use of the compositions comprising such peptides to new indications, and allow preventive intervention in conditions associated with, for example, tissue injury.

Conditions Associated with LRP1

LRP1 functions as an endocytic and cell signal transduction receptor and has several ligands that induce specific cell signaling cascades that can contribute to cell survival and anti-inflammatory mechanisms.^{5,18,22,25} LRP1 is ubiquitously expressed on many different organs, abundantly in brain, lung, heart, and immune cells. Because of these unique capabilities and wide expression on both tissues and immune cells, it plays a critical role in regulating inflammation, cellular metabolism, and maintaining homeostasis. For instance, LRP1 regulates inflammatory signaling pathways, such as NFκB and JNK pathways, that induce the conversion of pro-inflammatory (M1) macrophages to the anti-inflammatory (M2) macrophage phenotype, regulates the cytokine output, and contributes to effective migration and phagocytosis.^{22,26,51} In neutrophils, LRP1-dependent mechanisms lead to enhanced cell adhesion, chemotaxis, and antibacterial effects of these cells, thereby resisting immunosuppression²⁵. During acute infection or injury, LRP1 also promotes inflammatory resolution through scavenging PAMPS and DAMPS from dying or injured tissue, to prevent the tissue injury cycle²⁵. LRP1 was also shown to mediate autophagy during infection, an important metabolic process recently shown to play an important protective role in a variety of diseases.^{4,10} Therefore, because of its multifunctional ability to regulate inflammation, targeting LRP1 has substantial potential to mitigate several aspects of the immune response that contributes to the pathology of several diseases including neurological disorders, infectious diseases, and allergic inflammatory disease.

Neurological Disease

Peripheral nerve injury (PNI) resulting from metabolic, chemotherapy, or trauma often results in chronic pain. Neuropathic pain is characterized by evoked (allodynia, hyperalgesia) and spontaneous pain-like symptoms. The symptoms may be severe, including paresthesia, tingling, numbness, and burning sensations. Other than short term symptomatic relief, few therapeutic options are available and include steroids, local anesthetics, antidepressants, anti-seizure drugs, and opioids, which are reserved for severe pain. All these treatments aim at temporarily reducing pain to manageable levels; however, all can cause side effects and addiction and do not promote healing of damaged nerves. Accordingly, there is an unmet clinical need for novel and innovative pain treatments to prevent the transition from acute to chronic pain.

In terms of nerve injury and associated pain, injury to the peripheral nervous system induces an increase in the expression of LRP1. Previously, it was demonstrated that LRP1 agonists are capable of promoting axonal growth in the CNS and are capable of inducing regeneration after spinal cord injury.⁵³ LRP1 is an endocytic receptor to a diverse number of ligands, including tissue-type plasminogen activator (tPA), matrix metalloproteinase-9 (MMP-9), and activated α2-macroglobulin.¹⁴ These ligands are capable of inducing anti-inflammatory activity,³⁹ activating the Schwann cell repair program,²¹ and transactivation of cell signaling pathways in neurons associated with axonal regeneration.³⁸ LRP1 requires ligand-binding to activate cell-signaling, however, different ligands elicit distinct and sometimes opposing cell-signaling responses reflecting the ability of different ligands to assemble unique co-receptor complexes. Furthermore, many LRP1 ligands are multi-domain proteins with numerous effects on cell physiology that do not involve LRP1-binding. For example, tissue-type plasminogen activator (tPA) binds to LRP1 to promote Schwann cell (SC) survival and migration.²³ Yet, by LRP1-independent activities, tPA elicits pain.¹ EI-tPA promotes survival of human iPSC-derived neural progenitor cells (iNPCs) and transplanted EI-tPA activated iNPCs into rodents with severe spinal cord injury demonstrate improved motor functional recovery.⁴⁰ Imbalances in the microenvironment following nerve injury may have severe consequences, including the development of chronic neuropathic pain states.¹² In peripheral nervous system (PNS) injury, both inflammatory cytokines, such as TNFα, IL-6 and IL-1β, and anti-inflammatory cytokines, such as IL-10, have been shown to play a central role in axon regeneration and repair.⁶

Eosinophilic Driven Diseases (EDD)

Eosinophilic driven diseases (EDDs) are diseases associated with Type 2 inflammatory response accompanied with elevated levels of eosinophils and eosinophilic driven immune dysfunction. EDDs include EoE, eosinophilic asthma, atopic dermatitis, nasal polyps, and chronic spontaneous urticaria.

The peptides disclosed herein can serve as an adjunct treatment to mitigate the acute inflammatory response in patients with ST segment elevation myocardial infarction (STEMI). Additionally, SP163M has been shown to be safe and well tolerated in both Phase I and the ongoing Phase IIa clinical trials. The peptide therapy is unique in that it rebalances dysregulated immune responses and protects tissues from injury without any major immunosuppressive effects. Therefore, it is a safe anti-inflammatory drug that has broad-spectrum utility across a wide variety of immune-mediated diseases.

EoE is a chronic, largely type-2 immune mediated allergic inflammatory response associated with esophageal dysfunction and disturbed epithelial barrier function. This food allergen-driven disease is characterized by eosinophil dominated inflammation and type-2 mediated immune responses that lead to esophageal damage. In the U.S., an estimated 150,000 patients (largely children) are currently suffering from this disorder that commonly causes esophageal pain, difficulty swallowing, food impaction, persistent heartburn, chest and abdominal pain, weight loss, nausea, vomiting, and failure to thrive. Currently, there are no FDA-approved therapeutics for the treatment of EoE, and management of the disease consists of diet restrictions, proton pump inhibitors, and corticosteroids. The current standard-of-care therapies do not adequately address the immune dysregulation that occurs in EoE and other allergic inflammatory diseases, such as asthma or atopic dermatitis.

EoE is characterized by a high number of eosinophils, proteases, cathelicidin, serine proteases, including the kallikreins (KLK5), as well thymic stromal lymphopoietin (TSLP)—a cytokine and master regulator of allergic type-2 inflammatory responses in the local environment.⁴¹ In esophageal epithelial cells, a loss of the function of a serine peptidase inhibitor, Kazal type 7-SPINK7, results in uncontrolled protease activity, release of pro-inflammatory cytokines, such as TNFα, CCL2, GM-CSF, IL-8, and CXCL10, and inflammation. Recently, it was found that the serine protease KLK-5, which is an important mediator of epithelial barrier function, is a direct target of SPINK7 and loss of SPINK7 mediates EoE pathogenesis largely through uncontrolled KLK-5 protease activity. Interestingly, the SERPIN Alpha-1 antitrypsin is capable of inhibiting KLK5 activity in vitro and allergen-induced esophageal eosinophilia in vivo. However, the mechanism of the activity is not yet clear and may involve LRP1, instead of or in addition to direct proteolytic inhibition.

Patients with eosinophilic asthma have lower levels of LRP1. In addition, LRP1 deletion specifically of CD11b and CD11c dendritic cells in mice results in heightened allergic inflammatory response in an allergic airway disease model.²⁹ Mice with LRP1 deletion had increased antigen uptake and suffered increased eosinophilic inflammation, allergic sensitization, Th2 mediated cytokine production, and a reduction in T-regulatory cells.²⁹ Therefore, LRP1 could aid in maintaining homeostasis of proteases/inhibitors in the esophageal environment, mediating the TH2 responses and inhibiting inflammatory signaling pathways (NFκB, JNK) resulting in repair of esophageal dysfunction.

### SERPIN Peptides and Pharmaceutical Compositions Comprising the Same

Disclosed herein are SERPIN peptides including isolated, synthetic peptides and derivatives thereof that specifically bind to LRP1. LRP1 is an endocytic scavenger receptor for numerous ligands which exert biologically distinct functions. The LRP1 protein consists of a smaller (85 kD, β chain) intracellular fragment which spans the cell membrane, non-covalently attached to an extracellular fragment (515 kD, α chain) which consists of ligand-binding-type repeats, responsible for the majority of ligand binding. In addition to its ability to mediate the endocytosis for various lipoproteins, protease/inhibitor complexes, viruses, matrix proteins, and growth factors via its extracellular domain, LRP1 interacts with various scaffolding and signaling proteins via its intracellular domain to mediate cell signaling. Due to the multifunctional capability (both endocytic and cell signaling control) of LRP1, it is implicated in a variety of biological functions, including cell growth/survival, homeostasis, cell metabolism, cytokine regulation, and trafficking foreign antigens. Therefore, LRP1 is implicated to play a role in a variety of diseases.

In some embodiments, the SERPIN peptide comprises the SP16 peptide (SEQ ID NO: 2). In certain embodiments, the SERPIN peptide disclosed herein is an analog or derivative of SP16 peptide, sharing at least 50%, at least 55%, at least 60%, at least 65%, at least 70%, at least 75%, at least 80%, at least 85%, at least 90%, or at least 95% identity to SP16. In certain embodiments, the SERPIN peptide disclosed herein is an analog or derivative of SP16 peptide, sharing at least 50% identity to SP16. In certain embodiments, the SERPIN peptide disclosed herein is an analog or derivative of SP16 peptide, sharing at least 55% identity to SP16. In certain embodiments, the SERPIN peptide disclosed herein is an analog or derivative of SP16 peptide, sharing at least 60% identity to SP16. In certain embodiments, the SERPIN peptide disclosed herein is an analog or derivative of SP16 peptide, sharing at least 65% identity to SP16. In certain embodiments, the SERPIN peptide disclosed herein is an analog or derivative of SP16 peptide, sharing at least 70% identity to SP16. In certain embodiments, the SERPIN peptide disclosed herein is an analog or derivative of SP16 peptide, sharing at least 75% identity to SP16. In certain embodiments, the SERPIN peptide disclosed herein is an analog or derivative of SP16 peptide, sharing at least 80% identity to SP16. In certain embodiments, the SERPIN peptide disclosed herein is an analog or derivative of SP16 peptide, sharing at least 85% identity to SP16. In certain embodiments, the SERPIN peptide disclosed herein is an analog or derivative of SP16 peptide, sharing at least 90% identity to SP16. In certain embodiments, the SERPIN peptide disclosed herein is an analog or derivative of SP16 peptide, sharing at least 95% identity to SP16.

In certain embodiments, the SERPIN peptide disclosed herein comprises, consists essentially of, or consists of a core sequence FNKPFVFLM (SEQ ID NO: 1) of the SP16 peptide which has a sequence of VKFNKPFVFLMIEQNTK (SEQ ID NO: 2). The core sequence includes the LRP1 binding site having a sequence of FVFLM. Surprisingly, when Met of the core sequence is replaced by NIe, the activities of the SERPIN peptides increased significantly. Accordingly, in certain embodiments, the SERPIN peptide disclosed herein comprises, consists essentially of, or consists of a core binding motif having a sequence of X1-N-X2-P-F-X3-X4-X5-X6, wherein X1 is R or F, X2 is K or R, X3 is V or L, X4 is F, V, or M, X5 is L, V, or I, and X6 is M, I, or NIe. In certain embodiments, the binding motif has a sequence of FNKPFVFLM (SEQ ID NO: 1), FNKPFVFL[NIe] (SEQ ID NO: 5), FNRPFLVVI (SEQ ID NO: 6), FNRPFLVV[NIe] (SEQ ID NO: 7), FNRPFLMII (SEQ ID NO: 8), or FNRPFLVI[NIe] (SEQ ID NO: 9). In certain embodiments, the binding motif has a sequence of SEQ ID NO: 1. In certain embodiments, the binding motif has a sequence of SEQ ID NO: 5. In certain embodiments, the binding motif has a sequence of SEQ ID NO: 6. In certain embodiments, the binding motif has a sequence of SEQ ID NO: 7. In certain embodiments, the binding motif has a sequence of SEQ ID NO: 8. In certain embodiments, the binding motif has a sequence of SEQ ID NO: 9. In certain embodiments, the SERPIN peptide disclosed herein comprises, consists essentially of, or consists of an LRP1 binding site having a sequence of F-X3-X4-X5-X6, wherein X3 is V or L, X4 is F, V, or M, X5 is L, V, or I, and X6 is M, I, or NIe. In certain embodiments, the LRP1 binding site has a sequence of FVFLM (SEQ ID NO: 3), FVFL[NIe] (SEQ ID NO: 10), FLVVI (SEQ ID NO: 11), FLVV[NIe](SEQ ID NO: 12), FLMII (SEQ ID NO: 13), or FLMI[NIe] (SEQ ID NO: 14). In certain embodiments, the binding motif has a sequence of SEQ ID NO: 3. In certain embodiments, the binding motif has a sequence of SEQ ID NO: 10. In certain embodiments, the binding motif has a sequence of SEQ ID NO: 11. In certain embodiments, the binding motif has a sequence of SEQ ID NO: 12. In certain embodiments, the binding motif has a sequence of SEQ ID NO: 13. In certain embodiments, the binding motif has a sequence of SEQ ID NO: 14.

In certain embodiments, the SERPIN peptides disclosed herein comprise, consist essentially of, or consist of a modified core binding motif, by adding a flanking sequence comprising one or more basic amino acids, an arginine, or both of one or more basic amino acids and an arginine to either or both sides of the core binding motif. For example, the SERPIN peptide disclosed herein comprises, consists essentially of, or consists of an amino acid sequence of Z1-R-X1-N-X2-P-F-X3-X4-X5-X6-R-Z2, wherein X1 is R or F, X2 is K or R, X3 is V or L, X4 is F, V, or M, X5 is L, V, or I, X6 is M, I, or NIe, and Z1 and Z2 are independently 1, 2, 3, 4, 5, 6, 7, 8, 9, 10, or between 1 and 3, between 1 and 5, between 1 and 6, between 1 and 7, between 1 and 8, between 1 and 9, or between 1 and 10 basic amino acids. In certain embodiments, Z1 is 1 basic amino acid. In certain embodiments, Z1 is 2 basic amino acids. In certain embodiments, Z1 is 3 basic amino acids. In certain embodiments, Z1 is 4 basic amino acids. In certain embodiments, Z1 is 5 basic amino acids. In certain embodiments, Z1 is 6 basic amino acids. In certain embodiments, Z1 is 7 basic amino acids. In certain embodiments, Z1 is 8 basic amino acids. In certain embodiments, Z1 is 9 basic amino acids. In certain embodiments, Z1 is 10 basic amino acids. In certain embodiments, Z2 is 1 basic amino acid. In certain embodiments, Z2 is 2 basic amino acids. In certain embodiments, Z2 is 3 basic amino acids. In certain embodiments, Z2 is 4 basic amino acids. In certain embodiments, Z2 is 5 basic amino acids. In certain embodiments, Z2 is 6 basic amino acids. In certain embodiments, Z2 is 7 basic amino acids. In certain embodiments, Z2 is 8 basic amino acids. In certain embodiments, Z2 is 9 basic amino acids. In certain embodiments, Z2 is 10 basic amino acids. In certain embodiments, Z1 is between 1 and 3 basic amino acids. In certain embodiments, Z1 is between 1 and 5 basic amino acids. In certain embodiments, Z1 is between 1 and 6 basic amino acids. In certain embodiments, Z1 is between 1 and 7 basic amino acids. In certain embodiments, Z1 is between 1 and 8 basic amino acids. In certain embodiments, Z1 is between 1 and 9 basic amino acids. In certain embodiments, Z1 is between 1 and 10 basic amino acids. In certain embodiments, Z2 is between 1 and 3 basic amino acids. In certain embodiments, Z2 is between 1 and 5 basic amino acids. In certain embodiments, Z2 is between 1 and 6 basic amino acids. In certain embodiments, Z2 is between 1 and 7 basic amino acids. In certain embodiments, Z2 is between 1 and 8 basic amino acids. In certain embodiments, Z2 is between 1 and 9 basic amino acids. In certain embodiments, Z2 is between 1 and 10 basic amino acids.

In certain embodiments, the SERPIN peptide disclosed herein comprises, consists essentially of, or consists of an amino acid sequence of X1-X1-X1-X1-X1-X1-X1-X1-X1-X1-RFNRPFLVVIR-X1-X1-X1-X1-X1-X1-X1-X1-X1-X1 (SEQ ID NO: 17), X1-X1-X1-X1-X1-X1-X1-X1-X1-X1-RFNRPFLMIIR-X1-X1-X1-X1-X1-X1-X1-X1-X1-X1 (SEQ ID NO: 18), or X1-X1-X1-X1-X1-X1-X1-X1-X1-X1-KFNKPFVFL(NIe)R-X1-X1-X1-X1-X1-X1-X1-X1-X1-X1 (SEQ ID NO: 19), wherein X1 is one basic amino acid or none.

In certain embodiments, the SERPIN peptide disclosed herein comprises, consists essentially of, or consists of an amino acid sequence of X1-Z1-X2-Z2-X3-Z3-F-V-F-L-X4-Z4 (SEQ ID NO: 20), wherein:
X1 is V or L;
X2 is R or F;
X3 is R or K;
X4 is M, NIe, or I;
Z1 is any amino acid;
Z2 is any amino acid;
Z3 is any amino acid, and
Z4 is a sequence of any 5 amino acids.

In some embodiments, this peptide comprises, consists essentially of, or consists of 20 or fewer amino acids.

In certain embodiments, the SERPIN peptide disclosed herein comprises, consists essentially of, or consists of an amino acid sequence of X1-Z1-X2-Z2-X3-Z3-F-X4-F-L-Z4-X5 (SEQ ID NO: 21), wherein:
X1 is V or L;
X2 is F or R;
X3 is K or R;
X4 is V, L, or M;
X5 is a sequence of any 5 amino acids;
Z1 is any amino acid;
Z2 is a sequence of any 2 amino acids;
Z3 is any amino acid; and
Z4 is M, NIe, or I.

In some embodiments, this peptide comprises, consists essentially of, or consists of 20 or fewer amino acids.

In certain embodiments, the SERPIN peptide disclosed herein comprises, consists essentially of, or consists of the sequence of VKFNKPFVFL(NIe)IEQNTK (SEQ ID NO: 35), VKFNKPFVFLM (SEQ ID NO: 25), LRFNRPFLVVI (SEQ ID NO: 29), VRFNRPFLMII (SEQ ID NO: 31), VKFNKPFVFL(NIe) (SEQ ID NO: 40), RFNRPFLVVIR (SEQ ID NO: 41), RFNRPFLMIIR (SEQ ID NO: 42), RFNKPFVFL(NIe)R (SEQ ID NO: 43), RRRFLVVIRRR (SEQ ID NO: 44), RRRFLMIIRRR (SEQ ID NO: 45), or RRRFVFL(NIe)RRR (SEQ ID NO: 46). In certain embodiments, the SERPIN peptide disclosed herein comprises, consists essentially of, or consists of the sequence of SEQ ID NO: 35. In certain embodiments, the SERPIN peptide disclosed herein comprises, consists essentially of, or consists of the sequence of SEQ ID NO: 25. In certain embodiments, the SERPIN peptide disclosed herein comprises, consists essentially of, or consists of the sequence of SEQ ID NO: 29. In certain embodiments, the SERPIN peptide disclosed herein comprises, consists essentially of, or consists of the sequence of SEQ ID NO: 31. In certain embodiments, the SERPIN peptide disclosed herein comprises, consists essentially of, or consists of the sequence of SEQ ID NO: 40. In certain embodiments, the SERPIN peptide disclosed herein comprises, consists essentially of, or consists of the sequence of SEQ ID NO: 41. In certain embodiments, the SERPIN peptide disclosed herein comprises, consists essentially of, or consists of the sequence of SEQ ID NO: 42. In certain embodiments, the SERPIN peptide disclosed herein comprises, consists essentially of, or consists of the sequence of SEQ ID NO: 43. In certain embodiments, the SERPIN peptide disclosed herein comprises, consists essentially of, or consists of the sequence of SEQ ID NO: 44. In certain embodiments, the SERPIN peptide disclosed herein comprises, consists essentially of, or consists of the sequence of SEQ ID NO: 45. In certain embodiments, the SERPIN peptide disclosed herein comprises, consists essentially of, or consists of the sequence of SEQ ID NO: 46.

In certain embodiments, the SERPIN peptide disclosed herein has a size of between 5 and 30 amino acids. For example, the SERPIN peptide may have a size of 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, the SERPIN peptide may have a size of 5 amino acids. In some embodiments, the SERPIN peptide may have a size of 6 amino acids. In some embodiments, the SERPIN peptide may have a size of 7 amino acids. In some embodiments, the SERPIN peptide may have a size of 8 amino acids. In some embodiments, the SERPIN peptide may have a size of 9 amino acids. In some embodiments, the SERPIN peptide may have a size of 10 amino acids. In some embodiments, the SERPIN peptide may have a size of 11 amino acids. In some embodiments, the SERPIN peptide may have a size of 12 amino acids. In some embodiments, the SERPIN peptide may have a size of 13 amino acids. In some embodiments, the SERPIN peptide may have a size of 14 amino acids. In some embodiments, the SERPIN peptide may have a size of 15 amino acids. In some embodiments, the SERPIN peptide may have a size of 16 amino acids. In some embodiments, the SERPIN peptide may have a size of 17 amino acids. In some embodiments, the SERPIN peptide may have a size of 18 amino acids. In some embodiments, the SERPIN peptide may have a size of 19 amino acids. In some embodiments, the SERPIN peptide may have a size of 20 amino acids. In some embodiments, the SERPIN peptide may have a size of 21 amino acids. In some embodiments, the SERPIN peptide may have a size of 22 amino acids. In some embodiments, the SERPIN peptide may have a size of 23 amino acids. In some embodiments, the SERPIN peptide may have a size of 24 amino acids. In some embodiments, the SERPIN peptide may have a size of 25 amino acids. In some embodiments, the SERPIN peptide may have a size of 26 amino acids. In some embodiments, the SERPIN peptide may have a size of 27 amino acids. In some embodiments, the SERPIN peptide may have a size of 28 amino acids. In some embodiments, the SERPIN peptide may have a size of 29 amino acids. In some embodiments, the SERPIN peptide may have a size of 30 amino acids. In some embodiments, the SERPIN peptide disclosed herein has a size of 20 amino acids or less. A longer peptide may have a decreased solubility, whereas a shorter peptide may have decreased stability. As disclosed herein, various modifications can be made to improve stability, such as adding poly R sequences or other flanking sequences and forming a fusion protein.

The SERPIN peptides include analogues or derivatives thereof. For example, the native sequence of the SERPIN peptides can be modified to enhance plasma stability and result in an increased binding affinity to the peptide's cognate receptor. In certain embodiments, the SERPIN peptides disclosed herein can be further modified to extend the shelf life and/or bioavailability using one or more non-natural peptide bonds or amino acids or by attaching to the peptide functional groups such as polyethylene glycol (PEG). In certain embodiments, the SERPIN peptides disclosed herein are modified by adding one or more amino acid residues such as arginine at either or both ends. In certain embodiments, the SERPIN peptides are modified by adding two, three, or four amino acid residues at both ends. In some embodiments, the SERPIN peptides are modified by adding two amino acid residues at both ends. In some embodiments, the SERPIN peptides are modified by adding three amino acid residues at both ends. In some embodiments, the SERPIN peptides are modified by adding four amino acid residues at both ends.

In certain embodiments, the SERPIN peptides disclosed herein can have an N-terminus or C-terminus with additional or modified functional groups. In certain embodiments, one or both of the N-terminus and C-terminus of the SERPIN peptide may be amidated. In certain embodiments, the C-terminus of the SERPIN peptide may be amidated. In certain embodiments, the N-terminus of the SERPIN peptide may be amidated. In certain embodiments, one or both of the N-terminus and C-terminus of the SERPIN peptide may be acetylated.

In certain embodiments, the N-terminus of the SERPIN peptide may be acetylated. For example, in certain embodiments, the SERPIN peptide disclosed herein may comprise a sequence selected from a group consisting of Ac-VKFNKPFVFL(NIe)IEQNTK (N-terminal acetylated SEQ ID NO: 35), Ac-VKFNKPFVFLM (N-terminal acetylated SEQ ID NO: 25), Ac-LRFNRPFLVVI (N-terminal acetylated SEQ ID NO: 29), Ac-VRFNRPFLMII (N-terminal acetylated SEQ ID NO: 31), Ac-VKFNKPFVFL(NIe) (N-terminal acetylated SEQ ID NO: 40), Ac-RFNRPFLVVIR (N-terminal acetylated SEQ ID NO: 41), Ac-RFNRPFLMIIR (N-terminal acetylated SEQ ID NO: 42), Ac-RFNKPFVFL(NIe)R (N-terminal acetylated SEQ ID NO: 43), Ac-RRRFLVVIRRR (N-terminal acetylated SEQ ID NO: 44), Ac-RRRFLMIIRRR (N-terminal acetylated SEQ ID NO: 45), or Ac-RRRFVFL(NIe)RRR (N-terminal acetylated SEQ ID NO: 46).

In certain embodiments, the C-terminus of the SERPIN peptide may be amidated. For example, in certain embodiments, the SERPIN peptide disclosed herein may comprise a sequence selected from a group consisting of VKFNKPFVFL(NIe)IEQNTK-NH2 (C-terminal amidated SEQ ID NO: 35), VKFNKPFVFLM-NH2 (C-terminal amidated SEQ ID NO: 25), LRFNRPFLVVI-NH2 (C-terminal amidated SEQ ID NO: 29), VRFNRPFLMII-NH2 (C-terminal amidated SEQ ID NO: 31), VKFNKPFVFL(NIe)-NH2 (C-terminal amidated SEQ ID NO: 40), RFNRPFLVVIR-NH2 (C-terminal amidated SEQ ID NO: 41), RFNRPFLMIIR-NH2 (C-terminal amidated SEQ ID NO: 42), RFNKPFVFL(NIe)R-NH2 (C-terminal amidated SEQ ID NO: 43), RRRFLVVIRRR-NH2 (C-terminal amidated SEQ ID NO: 44), RRRFLMIIRRR-NH2 (C-terminal amidated SEQ ID NO: 45), or RRRFVFL(NIe)RRR-NH2 (C-terminal amidated SEQ ID NO: 46).

In certain embodiments, the C-terminus of the SERPIN peptide may be amidated and the N-terminus may be acetylated. For example, in certain embodiments, the SERPIN peptide disclosed herein may comprise a sequence selected from a group consisting of Ac-VKFNKPFVFL(NIe)IEQNTK-NH2 (C-terminal amidated and N-terminal acetylated SEQ ID NO: 35), Ac-VKFNKPFVFLM-NH2 (C-terminal amidated and N-terminal acetylated SEQ ID NO: 25), Ac-LRFNRPFLVVI-NH2 (C-terminal amidated and N-terminal acetylated SEQ ID NO: 29), Ac-VRFNRPFLMII-NH2 (C-terminal amidated and N-terminal acetylated SEQ ID NO: 31), Ac-VKFNKPFVFL(NIe)-NH2 (C-terminal amidated and N-terminal acetylated SEQ ID NO: 40), Ac-RFNRPFLVVIR-NH2 (C-terminal amidated and N-terminal acetylated SEQ ID NO: 41), Ac-RFNRPFLMIIR-NH2 (C-terminal amidated and N-terminal acetylated SEQ ID NO: 42), Ac-RFNKPFVFL(NIe)R-NH2 (C-terminal amidated and N-terminal acetylated SEQ ID NO: 43), Ac-RRRFLVVIRRR-NH2 (C-terminal amidated and N-terminal acetylated SEQ ID NO: 44), RRRFLMIIRRR-NH2 (C-terminal amidated and N-terminal acetylated SEQ ID NO: 45), or Ac-RRRFVFL(NIe)RRR-NH2 (C-terminal amidated and N-terminal acetylated SEQ ID NO: 46).

In certain embodiments, the SERPIN peptides disclosed herein are fused to one or more other peptides to form a fusion peptide or fusion protein. For example, one or more other peptides include an epitope tag, such as ALFA-tag, V5-tag, Myc-tag, HA-tag, Spot-tag, T7-tag, or NE-tag, a half-life extender, such as PEG, lipidation, FC fusion, or albumin fusion, or both of an epitope tag and a half-life extender. In certain embodiments, the peptide comprises one or more D-amino acids, that is, one or more amino acids of the peptide have a D-configuration.

In another aspect, this disclosure relates to a pharmaceutical composition comprising, consisting essentially of, or consisting of an effective amount of one or more SERPIN peptides or fusion peptides disclosed herein. In some embodiments, the pharmaceutical composition further comprises one or more additional therapeutic agents, which are not the SERPIN peptides disclosed herein. In some embodiments, the pharmaceutical composition further comprises a pharmaceutically acceptable carrier, excipient, additive, preservative, or a combination thereof. Examples of acceptable carriers include physiologically acceptable solutions, such as sterile saline and sterile buffered saline.

The term “an effective amount” as used herein refers to an amount of a composition that produces a desired effect. An effective amount of a composition may be used to produce a prophylactic or therapeutic effect in a subject, such as preventing or treating a target condition, alleviating symptoms associated with the condition, or producing a desired physiological effect. In such a case, the effective amount of a composition is a “therapeutically effective amount,” “therapeutically effective concentration” or “therapeutically effective dose.” The precise effective amount or therapeutically effective amount is an amount of the composition that will yield the most effective results in terms of efficacy of treatment in a given subject or population of cells. This amount will vary depending upon a variety of factors, including, but not limited to, the characteristics of the composition (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further, an effective or therapeutically effective amount may vary depending on whether the composition is administered alone or in combination with another composition, drug, therapy, or other therapeutic method or modality. One skilled in the clinical and pharmacological arts will be able to determine an effective amount or therapeutically effective amount through routine experimentation, namely by monitoring a cell's or subject's response to administration of a composition and adjusting the dosage accordingly. A “clinically effective amount,” “clinically effective concentration,” or “clinically effective dose” refers to a concentration or dose of a peptide, composition, or pharmaceutical composition that is shown to be effective in clinical trials or is predicted to be effective based on early phase or pre-clinical trials. For additional guidance, see Remington: The Science and Practice of Pharmacy, 21^{st} Edition, Univ. of Sciences in Philadelphia (USIP), Lippincott Williams & Wilkins, Philadelphia, PA, 2005.

In certain embodiments, the peptides or the pharmaceutical compositions disclosed herein may be formulated for oral administration, parenteral administration, such as intravenous administration, intramuscular administration, subcutaneous administration (bolus injection or through a device such as an infusion pump), intradermal administration, transdermal administration, topical administration, and intranasal administration. In certain embodiments, a subcutaneous infusion pump can be used for delivery of the peptides or the pharmaceutical compositions disclosed herein. The peptides or the pharmaceutical compositions may be administered more than once. More specifically, after the initial administration, one or more additional doses may be given as a booster.

Methods and Use of SERPIN Peptides and Pharmaceutical Compositions in Decreasing Inflammation, Treating Diseases and Conditions Associated with Pain, and Treating Diseases Associated with A. alternata.

The SERPIN peptides or the pharmaceutical compositions disclosed herein have various functions. In certain embodiments, disclosed herein is a method of treating a subject in need thereof an effective amount of one or more SERPIN peptides, fusion peptides, or the pharmaceutical compositions disclose herein. In some embodiments, the subject suffers from a disease or condition associated with LRP1 or TSLP. In some embodiments, the subject suffers from acute neuropathic pain, such as acute nociceptive, inflammatory, and neuropathic pain. In some embodiments, the subject suffers from an EDD, such as EoE, eosinophilic asthma, atopic dermatitis, nasal polyps, and chronic spontaneous urticaria. In some embodiments, the subject suffers from an allergic disease, allergic inflammation, or an eosinophil driven allergic disease.

As used herein, “treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. Treatment may also mean a prophylactic or preventative treatment of a condition.

As used herein, the term “subject” is a mammal. In some embodiments, the subject is a human. In some embodiments, the subject has not received any prior treatment with serine protease inhibitors, such as alpha-1-antitrypsin treatment, before the treatment with the peptides disclosed herein.

In some embodiments, the SERPIN peptides and fusions thereof or pharmaceutical compositions comprising the peptides or fusions can be administered at a dose from about 0.001 mg/kg to about 4 mg/kg in humans. Depending on the indication, severity, and administration route, a suitable dose can be selected accordingly. For example, for acute indications, fewer treatments with a higher dose in each treatment are administered; while for chronic indications requiring frequent and long-term treatment, a lower dose in each treatment is administered. In some indications where the inflamed tissue expresses a high density of LRP1, a very low dose of the SERPIN peptides such as SP16 and SP163M is required. When a subject suffers from a nerve injury, neurons express a very high density of LRP1. In vivo and in vitro studies demonstrated a significant effect with a low dose of 0.05 μg.

In some embodiments, the SERPIN peptides and fusions thereof or pharmaceutical compositions comprising the peptides or fusions is administered subcutaneously. In some embodiments, a SERPIN peptide, such as SP16 or SP163M, is administered subcutaneously to a human subject at a dose of between 0.05 mg/kg and 0.5 mg/kg, for example, at a dose of 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, or 0.5 mg/kg. In some embodiments, the SERPIN peptide is administered at a dose of 0.05 mg/kg. In some embodiments, the SERPIN peptide is administered at a dose of 0.1 mg/kg. In some embodiments, the SERPIN peptide is administered at a dose of 0.15 mg/kg. In some embodiments, the SERPIN peptide is administered at a dose of 0.2 mg/kg. In some embodiments, the SERPIN peptide is administered at a dose of 0.25 mg/kg. In some embodiments, the SERPIN peptide is administered at a dose of 0.3 mg/kg. In some embodiments, the SERPIN peptide is administered at a dose of 0.35 mg/kg. In some embodiments, the SERPIN peptide is administered at a dose of 0.4 mg/kg. In some embodiments, the SERPIN peptide is administered at a dose of 0.45 mg/kg. In some embodiments, the SERPIN peptide is administered at a dose of 0.5 mg/kg. In some embodiments, a SERPIN peptide, such as SP16 or SP163M, is administered subcutaneously to a human subject at a dose of 0.2 mg/kg or 0.4 mg/kg. In some embodiments, a SERPIN peptide, such as SP16 or SP163M, is administered orally to a human subject at a dose between 1 mg and 150 mg, for example, at a dose of 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, or 150 mg. In some embodiments, a SERPIN peptide, such as SP16 or SP163M, is administered orally to a human subject at a dose of 5 mg, 25 mg, or 100 mg. In some embodiments, a SERPIN peptide, such as SP16 or SP163M, is administered locally, for example, by a transdermal patch, optionally with pulsatile delivery, at a dose between 0.05 mg/kg and 0.5 mg/kg, for example, at a dose of 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, or 0.5 mg/kg.

In some embodiments, a single dose of the SERPIN peptides and fusions thereof or pharmaceutical compositions comprising the peptides or fusions is administered. In some embodiments, the SERPIN peptides and fusions thereof or pharmaceutical compositions comprising the peptides or fusions is administered as the sole therapeutic agent. In some embodiments, the SERPIN peptides and fusions thereof or pharmaceutical compositions comprising the peptides or fusions is administered in combination with a secondary therapeutic agent.

In some embodiments, the SERPIN peptides and fusions thereof or pharmaceutical compositions comprising the peptides or fusions is administered in a pulsatile mode or a continuous mode. In some embodiments, the SERPIN peptides and fusions thereof or pharmaceutical compositions comprising the peptides or fusions is administered via a transdermal patch, an inhaler, or an intranasal device.

Methods of Reducing Inflammation in a Subject Having a Disease or Condition Associated with LRP1 or TSLP by Administering a SERPIN Peptide to a Subject in Need Thereof.

In some embodiments, the present technology includes a method of reducing inflammation in a subject having a disease or condition associated with LRP1 or TSLP. In some aspects, the method comprises administering any SERPIN peptide described in this disclosure to a subject in need thereof.

In some aspects, the SERPIN peptide that is administered to reduce inflammation in a subject having a disease or condition associated with LRP1 or TSLP comprises an amino acid sequence selected from the group consisting of VKFNKPFVFL(NIe)IEQNTK (SEQ ID NO: 35), VKFNKPFVFLMIEQNTK (SEQ ID NO: 2), VKFNKPFVFLM (SEQ ID NO: 25), LRFNRPFLWI (SEQ ID NO: 29), VRFNRPFLMII (SEQ ID NO: 31), VKFNKPFVFL(NIe) (SEQ ID NO: 40), RFNRPFLVVIR (SEQ ID NO: 41), RFNRPFLMIIR (SEQ ID NO: 42), RFNKPFVFL(NIe)R (SEQ ID NO: 43), RRRFLVVIRRR (SEQ ID NO: 44), RRRFLMIIRRR (SEQ ID NO: 45), RRRFVFL(NIe)RRR (SEQ ID NO: 46), FVFLM (SEQ ID NO: 3), and FVFL(NIe) (SEQ ID NO: 10).

In some aspects, the SERPIN peptide that is administered to reduce inflammation in a subject having a disease or condition associated with LRP1 or TSLP comprises the SP16 peptide (SEQ ID NO: 2) or the SP163M peptide (SEQ ID NO: 35). In some aspects, the SERPIN peptide comprises an amino acid sequence that shares at least 95% identity with the SP16 peptide (SEQ ID NO: 2). In some aspects, the SERPIN peptide comprises an amino acid sequence that shares at least 95% identity with the SP163M peptide (SEQ ID NO: 35). In some aspects, the SERPIN peptide comprises an amino acid sequence that shares at least 90% identity with the SP16 peptide (SEQ ID NO: 2). In some aspects, the SERPIN peptide comprises an amino acid sequence that shares at least 90% identity with the SP163M peptide (SEQ ID NO: 35).

In some aspects, the SERPIN peptide that is administered to reduce inflammation in a subject having a disease or condition associated with LRP1 or TSLP comprises an amino acid sequence of X1-N-X2-P-F-X3-X4-X5-X6, wherein X1 is R or F, X2 is K or R, X3 is V or L, X4 is F, V, or M, X5 is L, V, or I, and X6 is M, I, or NIe. In some aspects, the SERPIN peptide comprises a sequence of FNKPFVFLM (SEQ ID NO: 1), FNKPFVFL[NIe] (SEQ ID NO: 5), FNRPFLVVI (SEQ ID NO: 6), FNRPFLVV[NIe] (SEQ ID NO: 7), FNRPFLMII (SEQ ID NO: 8), or FNRPFLVI[NIe] (SEQ ID NO: 9).

In some aspects, the SERPIN peptide that is administered to reduce inflammation in a subject having a disease or condition associated with LRP1 or TSLP comprises an amino acid sequence of X1-Z1-X2-Z2-X3-Z3-F-V-F-L-X4-Z4 (SEQ ID NO: 20), wherein: X1 is V or L; X2 is R or F; X3 is R or K; X4 is M, NIe, or I; Z1 is any amino acid; Z2 is any amino acid; Z3 is any amino acid; and Z4 is a sequence of any five amino acids. In some aspects, the SERPIN peptide comprises an amino acid sequence of X1-Z1-X2-Z2-X3-Z3-F-X4-F-L-Z4-X5 (SEQ ID NO: 21), wherein: X1 is V or L; X2 is F or R; X3 is K or R; X4 is V, L, or M; X5 is a sequence any five amino acids; Z1 is any amino acid; Z2 is a sequence of any two amino acids; Z3 is any amino acid; and Z4 is M, NIe, or I.

In some aspects, the SERPIN peptide that is administered to reduce inflammation in a subject having a disease or condition associated with LRP1 or TSLP comprises the sequence of SEQ ID NO: 35. In some aspects, the amino acid sequence of the SERPIN peptide comprises the sequence of SEQ ID NO: 2. In some aspects, the N-terminus of the SERPIN peptide is acetylated. In some aspects, the C-terminus of the SERPIN peptide is amidated. In some aspects, the SERPIN peptide is fused to one or more other peptides to form a fusion peptide or fusion protein. In some aspects, the one or more other peptides is different from the SERPIN peptide. In some aspects, the fusion peptide or fusion protein comprises the SERPIN peptide and an epitope tag, a half-life extender, or both the epitope tag and the half-life extender. In some aspects, the technology includes a pharmaceutical composition comprising the SERPIN peptide and a pharmaceutically effective carrier.

In some embodiments, the SERPIN peptide that is administered to reduce inflammation in a subject having a disease or condition associated with LRP1 or TSLP is administered at a therapeutically effective dose or concentration. In some aspects, the pharmaceutical compound is administered at a therapeutically effective dose. In some aspects, the SERPIN peptide is administered at a clinically effective dose or concentration. In some aspects, the pharmaceutical compound is administered at a clinically effective dose. In some aspects, the SERPIN peptide is administered at a dose of between 0.001 mg/kg and 5 mg/kg. In some aspects, the subject is a human. In some aspects, the administration is by oral administration, parenteral administration, intradermal administration, transdermal administration, topical administration, or intranasal administration. In some aspects, the composition is administered as a single dose. In some aspects, the SERPIN peptide is administered by topical administration. In some aspects, the SERPIN peptide is administered by oral administration.

In some aspects, the SERPIN peptide that is administered to reduce inflammation in a subject having a disease or condition associated with LRP1 or TSLP, wherein the disease or condition is caused by A. alternata. In some aspects, the disease or condition is rhinitis, asthma, dermatitis, or esophageal eosinophilia. In some aspects, the disease or condition is rhinitis. In some aspects, the disease or condition is asthma. In some aspects, the disease or condition is dermatitis. In some aspects, the disease or condition is esophageal eosinophilia. In some aspects, the disease or condition is an acute or neuropathic pain. In some aspects, the disease or condition is acute nociceptive, inflammatory, or neuropathic pain. In some aspects, the disease or condition is an EDD. In some aspects, the disease or condition is EoE, eosinophilic asthma, atopic dermatitis, nasal polyps, or chronic spontaneous urticaria. In some aspects, the disease or condition is atopic dermatitis. In certain embodiments, the disease or condition is pruritis. In some aspects, the disease or condition is an allergic reaction. In some aspects, the disease or condition is allergic inflammation. In some aspects, the disease or condition is an eosinophilic driven allergic disease. In some aspects, the disease or condition is caused by TH2 driven inflammatory cytokines.

### Methods for Treating Acute or Neuropathic Pain, Nociceptive Pain, or Inflammatory Pain by Administering a SERPIN Peptide to a Subject in Need Thereof.

In some embodiments, the present technology includes a method of treating a subject having a disease or condition associated with LRP1 or TSLP, where the disease or condition is acute or neuropathic pain, nociceptive pain, or inflammatory pain. In some aspects, the present technology includes a method of treating a subject having a disease or condition associated with LRP1, where the disease or condition is acute or neuropathic pain, nociceptive pain, or inflammatory pain. In some aspects, the method comprises administering any SERPIN peptide described in this disclosure to a subject in need thereof.

In some aspects, the method of treating acute or neuropathic pain, nociceptive pain, or inflammatory pain comprises administering a SERPIN peptide that comprises an amino acid sequence selected from the group consisting of VKFNKPFVFL(NIe)IEQNTK (SEQ ID NO: 35), VKFNKPFVFLMIEQNTK (SEQ ID NO: 2), VKFNKPFVFLM (SEQ ID NO: 25), LRFNRPFLVVI (SEQ ID NO: 29), VRFNRPFLMII (SEQ ID NO: 31), VKFNKPFVFL(NIe) (SEQ ID NO: 40), RFNRPFLVVIR (SEQ ID NO: 41), RFNRPFLMIIR (SEQ ID NO: 42), RFNKPFVFL(NIe)R (SEQ ID NO: 43), RRRFLVVIRRR (SEQ ID NO: 44), RRRFLMIIRRR (SEQ ID NO: 45), RRRFVFL(NIe)RRR (SEQ ID NO: 46), FVFLM (SEQ ID NO: 3), and FVFL(NIe) (SEQ ID NO: 10). In some aspects, the SERPIN peptide is administered to the subject to treat the disease or condition associated with LRP1 or TSLP. In some aspects, the SERPIN peptide is administered to the subject to treat the disease or condition associated with LRP1.

In some aspects, the method of treating acute or neuropathic pain, nociceptive pain, or inflammatory pain comprises administering a SERPIN peptide that comprises the SP16 peptide (SEQ ID NO: 2) or the SP163M peptide (SEQ ID NO: 35). In some aspects, the SERPIN peptide comprises an amino acid sequence that shares at least 95% identity with the SP16 peptide (SEQ ID NO: 2). In some aspects, the SERPIN peptide comprises an amino acid sequence that shares at least 95% identity with the SP163M peptide (SEQ ID NO: 35). In some aspects, the SERPIN peptide comprises an amino acid sequence that shares at least 90% identity with the SP16 peptide (SEQ ID NO: 2). In some aspects, the SERPIN peptide comprises an amino acid sequence that shares at least 90% identity with the SP163M peptide (SEQ ID NO: 35).

In some aspects, the method of treating acute or neuropathic pain, nociceptive pain, or inflammatory pain comprises administering a SERPIN peptide that comprises an amino acid sequence of X1-N-X2-P-F-X3-X4-X5-X6, wherein X1 is R or F, X2 is K or R, X3 is V or L, X4 is F, V, or M, X5 is L, V, or I, and X6 is M, I, or NIe. In some aspects, the SERPIN peptide comprises a sequence of FNKPFVFLM (SEQ ID NO: 1), FNKPFVFL[NIe](SEQ ID NO: 5), FNRPFLVVI (SEQ ID NO: 6), FNRPFLVV[NIe] (SEQ ID NO: 7), FNRPFLMII (SEQ ID NO: 8), or FNRPFLVI[NIe] (SEQ ID NO: 9).

In some aspects, the method of treating acute or neuropathic pain, nociceptive pain, or inflammatory pain comprises administering a SERPIN peptide comprising an amino acid sequence of X1-Z1-X2-Z2-X3-Z3-F-V-F-L-X4-Z4 (SEQ ID NO: 20), wherein: X1 is V or L; X2 is R or F; X3 is R or K; X4 is M, NIe, or I; Z1 is any amino acid; Z2 is any amino acid; Z3 is any amino acid; and Z4 is a sequence any five amino acids. In some aspects, the SERPIN peptide comprises of an amino acid sequence of X1-Z1-X2-Z2-X3-Z3-F-X4-F-L-Z4-X5 (SEQ ID NO: 21), wherein: X1 is V or L; X2 is F or R; X3 is K or R; X4 is V, L, or M; X5 is a sequence any five amino acids; Z1 is any amino acid; Z2 is a sequence of any two amino acids; Z3 is any amino acid; and Z4 is M, NIe, or I.

In some aspects, the method of treating acute or neuropathic pain, nociceptive pain, or inflammatory pain comprises administering a SERPIN peptide that comprises the sequence of SEQ ID NO: 35. In some aspects, the amino acid sequence of the SERPIN peptide comprises the sequence of SEQ ID NO: 2. In some aspects, the N-terminus of the SERPIN peptide is acetylated. In some aspects, the C-terminus of the SERPIN peptide is amidated. In some aspects, the SERPIN peptide is fused to one or more other peptides to form a fusion peptide or fusion protein. In some aspects, the one or more other peptides is different from the SERPIN peptide. In some aspects, the fusion peptide or fusion protein comprises the SERPIN peptide and an epitope tag, a half-life extender, or both the epitope tag and the half-life extender. In some aspects, the technology includes a pharmaceutical composition comprising the SERPIN peptide and a pharmaceutically effective carrier.

In some aspects, the method of treating acute or neuropathic pain, nociceptive pain, or inflammatory pain comprises administering the SERPIN peptide at a therapeutically effective dose or concentration. In some aspects, the pharmaceutical compound is administered at a therapeutically effective dose. In some aspects, the SERPIN peptide is administered at a clinically effective dose or concentration. In some aspects, the pharmaceutical compound is administered at a clinically effective dose. In some aspects, the SERPIN peptide is administered at a dose of between 0.001 mg/kg and 5 mg/kg. In some aspects, the subject is a human. In some aspects, the administration is by oral administration, parenteral administration, intradermal administration, transdermal administration, topical administration, or intranasal administration. In some aspects, the composition is administered as a single dose. In some aspects, the SERPIN peptide is administered by topical administration. In some aspects, the SERPIN peptide is administered by oral administration.

In some aspects, the method of treating acute or neuropathic pain, nociceptive pain, or inflammatory pain comprises administering the SERPIN peptide to treat a disease or condition associated with LRP1. In some aspects, the disease or condition is acute or neuropathic pain. In some aspects, the disease or condition is nociceptive pain. In some aspects, the disease or condition is inflammatory pain. In some aspects, administering the SERPIN peptide results in reduced pain. In some aspects, administering the SERPIN peptide prevents or reduces the development of pain. In some aspects, administering the SERPIN peptide results in increased neuronal survival and neurite sprouting.

Methods for Treating Diseases or Conditions Caused by A. alternata by Administering a SERPIN Peptide to a Subject in Need Thereof.

In some embodiments, the present technology includes a method of treating a subject having a disease or condition associated with LRP1 or TSLP, where the disease or condition is caused by A. alternata. In some aspects, the present technology includes a method of treating a subject having a disease or condition associated with TSLP, where the disease or condition is caused by A. alternata. In some aspects, the method comprises administering any SERPIN peptide described in this disclosure to a subject having a disease or condition caused by A. alternata.

In certain embodiments, the method of treating a disease or condition caused by A. alternata comprises administering a SERPIN peptide comprising an amino acid sequence selected from the group consisting of VKFNKPFVFL(NIe)IEQNTK (SEQ ID NO: 35), VKFNKPFVFLMIEQNTK (SEQ ID NO: 2), VKFNKPFVFLM (SEQ ID NO: 25), LRFNRPFLVVI (SEQ ID NO: 29), VRFNRPFLMII (SEQ ID NO: 31), VKFNKPFVFL(NIe) (SEQ ID NO: 40), RFNRPFLVVIR (SEQ ID NO: 41), RFNRPFLMIIR (SEQ ID NO: 42), RFNKPFVFL(NIe)R (SEQ ID NO: 43), RRRFLVVIRRR (SEQ ID NO: 44), RRRFLMIIRRR (SEQ ID NO: 45), RRRFVFL(NIe)RRR (SEQ ID NO: 46), FVFLM (SEQ ID NO: 3), and FVFL(NIe) (SEQ ID NO: 10) to the subject to treat the disease or condition caused by A. alternata.

In some aspects, the method of treating a disease or condition caused by A. alternata comprises administering a SERPIN peptide comprising the SP16 peptide (SEQ ID NO: 2) or the SP163M peptide (SEQ ID NO: 35). In some aspects, the SERPIN peptide comprises an amino acid sequence that shares at least 95% identity with the SP16 peptide (SEQ ID NO: 2). In some aspects, the SERPIN peptide comprises an amino acid sequence that shares at least 95% identity with the SP163M peptide (SEQ ID NO: 35). In some aspects, the SERPIN peptide comprises an amino acid sequence that shares at least 90% identity with the SP16 peptide (SEQ ID NO: 2). In some aspects, the SERPIN peptide comprises an amino acid sequence that shares at least 90% identity with the SP163M peptide (SEQ ID NO: 35).

In some aspects, the method of treating a disease or condition caused by A. alternata comprises administering a SERPIN peptide comprising an amino acid sequence of X1-N-X2-P-F-X3-X4-X5-X6, wherein X1 is R or F, X2 is K or R, X3 is V or L, X4 is F, V, or M, X5 is L, V, or I, and X6 is M, I, or NIe. In some aspects, the SERPIN peptide comprises a sequence of FNKPFVFLM (SEQ ID NO: 1), FNKPFVFL[NIe] (SEQ ID NO: 5), FNRPFLVVI (SEQ ID NO: 6), FNRPFLVV[NIe] (SEQ ID NO: 7), FNRPFLMII (SEQ ID NO: 8), or FNRPFLVI[NIe] (SEQ ID NO: 9).

In some aspects, the method of treating a disease or condition caused by A. alternata comprises administering a SERPIN peptide comprising an amino acid sequence of X1-Z1-X2-Z2-X3-Z3-F-V-F-L-X4-Z4 (SEQ ID NO: 20), wherein: X1 is V or L; X2 is R or F; X3 is R or K; X4 is M, NIe, or I; Z1 is any amino acid; Z2 is any amino acid; Z3 is any amino acid; and Z4 is a sequence any five amino acids. In some aspects, the SERPIN peptide comprises of an amino acid sequence of X1-Z1-X2-Z2-X3-Z3-F-X4-F-L-Z4-X5 (SEQ ID NO: 21), wherein: X1 is V or L; X2 is F or R; X3 is K or R; X4 is V, L, or M; X5 is a sequence any five amino acids; Z1 is any amino acid; Z2 is a sequence of any two amino acids; Z3 is any amino acid; and Z4 is M, NIe, or I.

In certain embodiments, the method of treating a disease or condition caused by A. alternata comprises administering a SERPIN peptide comprising the amino acid sequence of SEQ ID NO: 35. In some aspects, the amino acid sequence of the SERPIN peptide comprises the sequence of SEQ ID NO: 2. In certain embodiments, the N-terminus of the SERPIN peptide is acetylated. In certain embodiments, the C-terminus of the SERPIN peptide is amidated. In certain embodiments, the SERPIN peptide is fused to one or more other peptides to form a fusion peptide or fusion protein. In certain embodiments, the one or more other peptides is different from the SERPIN peptide. In certain embodiments, the fusion peptide or fusion protein comprises the SERPIN peptide and an epitope tag, a half-life extender, or both the epitope tag and the half-life extender. In some aspects, the technology includes a pharmaceutical composition comprising the SERPIN peptide and a pharmaceutically effective carrier.

In some aspects, the method of treating a disease or condition caused by A. alternata comprises administering a SERPIN peptide at a therapeutically effective dose or concentration. In some aspects, the pharmaceutical compound is administered at a therapeutically effective dose. In some aspects, the SERPIN peptide is administered at a clinically effective dose or concentration. In some aspects, the pharmaceutical compound is administered at a clinically effective dose. In some aspects, the SERPIN peptide is administered at a dose of between 0.001 mg/kg and 5 mg/kg. In some aspects, the subject is a human. In some aspects, the administration is by oral administration, parenteral administration, intradermal administration, transdermal administration, topical administration, or intranasal administration. In some aspects, the SERPIN peptide is administered as a single dose.

In some aspects, the method of treating a disease or condition caused by A. alternata comprises administering a SERPIN peptide, wherein the disease or condition is rhinitis, asthma, dermatitis, or esophageal eosinophilia. In some aspects, the disease or condition is rhinitis. In some aspects, the disease or condition is asthma. In some aspects, the disease or condition is dermatitis. In certain embodiments, the disease or condition is esophageal eosinophilia. In some aspects, administering the SERPIN peptide reduces inflammation. In some aspects, administering the SERPIN peptide reduces eosinophilic inflammation.

### EXAMPLES

The following examples are intended to illustrate various embodiments, of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Materials and Methods

Animals: Male Sprague Dawley rats (170-200 g; 8-12 weeks old) and C57BL/6J mice (25 g; male and female, 8-12 weeks old) were purchased from Envigo and Jackson Laboratory, respectively. All animal experiments were approved by the Institutional Animal Care and Use Committee at University of California, San Diego. All rats and mice were housed with a 12 h:12 h light: dark cycle with ad libitum access to food and water.

Reagents: SP163M (Ac-VKFNKPFVFLNIeIEQNTK-NH2; SEQ ID NO: 35) was provided from Serpin Pharma (Manassas, VA, USA). Briefly, peptides were synthesized by CPC Scientific Inc (Sunnyvale, CA) with purity >95% as verified by high performance liquid chromatography and mass spectroscopy. Recombinant human EI-tPA was purchased from Molecular Innovations (Novi, MI, USA). NGF-β was purchased from Sigma (St. Louis, MO, USA). Receptor associated protein was expressed as a glutathione-S-transferase (GST)-fusion protein (GST-RAP).

Neurite outgrowth in primary cultures of adult DRG neurons: Primary DRGs neurons were isolated from adult male Sprague Dawley rats and cultured as previously described for mice with modifications.⁵³ The DRGs were stripped of their roots and collected in Hanks Buffered Salt Solution (HBSS) on ice. DRGs were enzymatically digested and approximately 4000 DRG neurons were plated in each well of a 12-well tissue culture plate (Thermo Fisher Scientific, Waltham, MA, USA). All DRG neurons were cultured at 37° C. in 5% CO₂ for 54 h in DMEM/F12 containing 2% B27 and 1% FBS with vehicle or SP163M (0-500 ng/ml) added every 24 h. Primary cultured DRG neurons were imaged by phase contrast and the viability of cells was assessed by Trypan blue. Primary DRG neurons were cultured, fixed in 4% paraformaldehyde, and immunofluorescence was performed using a mouse anti-βIII-tubulin primary antibody (Promega, Madison, WI, USA; cat #G7121, 1:250) and then with Alexa Fluor-488 anti-mouse antibody (Life Technologies, Carlsbad, CA, USA) as secondary antibody. DRG neurons were imaged at 20× and 40× manually, and the longest neurite length per cell was measured in 11 images from multiple wells and separate experiments. Approximately 222 and 144 neurons were measured in SP16 and control groups, respectively. Quantification was performed in a blinded manner. For all neurite outgrowth measurements, at least 6 individual experiments were performed in duplicate.

Cell signaling analysis: Rat PC12 cells were purchased from ATCC (CRL-1721). PC12 cells were maintained in high glucose DMEM (Gibco, USA) containing 10% heat-inactivated FBS (Gibco, USA), 5% HyClone heat-inactivated horse serum (Cytiva, USA), penicillin (100 units/ml) and streptomycin (1 mg/ml) in 6-well plates that were pre-coated with 0.01 mg/ml type IV collagen (Sigma-Aldrich, St. Louis, MO, USA). Cells were transferred to serum-free medium (SFM) 4 h prior to adding effectors, and then treated with SP163M (2.4, 24 or 240 nM); EI-tPA (12 nM), NGF (0.36 nM), or vehicle (PBS) for 10 min. In some cases, cells were pre-incubated with the competitive antagonist of LRP1, GST-RAP (150 nM) for 15 min or electroporated using the Rat Neuron Nucleofector Amaxa Kit (Lonza Biosciences) and incubated with siRNA to silence LRP1 expression (siLRP1; M-094191-01-0010, Dharmacon) for 48 h. Control cells were transfected with non-targeting control (NTC) siRNA (NTC; D-001810-10-05, Dharmacon). Cells were rinsed with ice-cold PBS and proteins were extracted in RIPA buffer (20 mM sodium phosphate, 150 mM NaCl, pH 7.4, 1% Triton X-100, 0.5% sodium deoxycholate, 0.1% SDS) supplemented with protease and phosphatase inhibitors (Roche Diagnostics, USA). After 30 min on ice, lysates were centrifuged at 15000×g for 5 min, supernatant collected and stored at −20° C. Equal amounts of protein from cell lysates (20 μg), as determined by BCA Protein Assay (ThermoFisher Scientific, Waltham, MA, USA), were subjected to 10% SDS-PAGE and electro-transferred to nitrocellulose membranes. The membranes were blocked with 5% nonfat dried milk and then incubated with primary anti-phospho-AKT (Cell Signaling Technology, Danvers, MA, USA; cat #9271S; 1:1000), anti-phospho-ERK1/2 (Cell Signaling Technology, Danvers, MA, USA; cat #9101S; 1:1000), anti-LRP1 (Cell Signaling Technology, Danvers, MA, USA; cat #64099S; 1:1000) or anti total-ERK1/2 (Cell Signaling Technology, Danvers, MA, USA; cat #91012S; 1:1000). Immunoblots were developed using Radiance, Radiance Q, and Radiance Plus chemiluminescent substrates and imaged using a BioRad ChemiDoc Imaging System (Bio-Rad, Hercules, CA, USA).

RT-qPCR: RNA was isolated from DRG cultures using the NucleoSpin RNA kit (MachereyNagel, Duren, GER) and reverse-transcribed using the iScript cDNA synthesis kit (Bio-Rad, Hercules, CA, USA). qPCR was performed using TaqMan gene expression products (ThermoFisher Scientific, Waltham, MA, USA) for GAP-43 (Rn01474579), LRP1 (Rn01503901_m1) and GAPDH (Rn99999916_s1). Amplification was performed with CFX Connect Real-Time PCR detection system (Bio-Rad, Hercules, CA, USA). The relative change in mRNA expression was calculated using the 2^{ΔΔCT} method with GAPDH mRNA as an internal normalizer as disclosed previously.⁵⁶

Intraplantar formalin and capsaicin models: Male mice (n=33) were acclimated to the behavior testing facility for at least 60 min. Mice were randomized into four groups SP163M (0.02, 0.2, and 2 μg/g) or vehicle were administered subcutaneously. After 1 h, 20 μl of 2.5% formalin was injected subcutaneously into the plantar area of left hind paw. Immediately after formalin injection, mice were placed in a Plexiglas box (22×22×14 cm). Two observers, blinded to treatments, recorded the total amount of time mice spent on licking and flinching the left hind paw every 5 min over a one-hour period. To quantify the formalin response, activity during early phase (0-10 min) and later phase (15-50 min) were examined separately.

For the capsaicin studies, male (n=41) and female (n=37) mice were acclimated to the behavior testing facility for at least 1 h. Capsaicin was dissolved in 20% (2-hydroxypropyl)-β-cyclodextrin (Sigma-Aldrich, St. Louis, MO, USA) solution. This vehicle concentration solubilized the capsaicin and did not induce a behavioral response when administered alone. One hour prior to intraplantar injections, vehicle, SP163M (2 μg/g; s.c.) or enzymatically inactive tPA (EI-tPA; 2 μg/g i.v.) were administered. Subsequently, 10 μl of 2 μg/μl capsaicin solution was injected into the plantar area of left hind paw. Immediately after capsaicin injection, mice were placed in a plexiglass box. Two observers, blinded to treatments, recorded the time spent licking and flinching the left hind paw over 10 min.

Neuropathic pain model: Mice (n=30) were randomly assigned to two different groups: SP163M (2 μg/g; s.c. 100 μl) and vehicle (H20 s.c. 100 μl). Mice were treated 1 h prior to partial nerve ligation (PNL) and then daily at least 1 h prior to behavior testing for 2 weeks. PNL studies were performed as previously disclosed⁵⁹ and adapted for mice.⁵⁷ Male mice were anesthetized with 3% isoflurane (Vetone, USA) in 1.5 L/min oxygen (Praxair, USA) and maintained with 2.5% isoflurane. An incision was made along the long axis of the femur. The sciatic nerve was exposed at mid-thigh level by separating the biceps femoris and the gluteus superficialis and then carefully cleared of surrounding connective tissue. A 9-0 nylon suture (Ethicon, Inc., Somerville, NJ, USA) was inserted into the nerve and ligated so that the one-third to one-half of the nerve was included. The muscle and skin layers were closed using Reflex7 7 mm stainless steel wound clips (CellPoint Scientific, Inc., Gaithersburg, MD, USA). For behavior testing, mice were acclimated, and baseline tested for one week prior to PNL. Mechanical sensitivity (tactile allodynia) was tested by applying 0.04 to 4 g Von Frey filaments (Stoelting, Wood Dale IL, USA) to the plantar surface of the ipsilateral hind paw. Filaments were presented in a consecutive fashion either ascending or descending using the up-down method as previously disclosed⁶⁰ and modified for mice.^{57,61} The filament that caused paw withdrawal 50% of the time (the 50% PWT) was determined. Tactile allodynia was tested on days 2, 4, 9, 11, and 14 days following PNL. Results were averaged and subjected to statistical analysis. All experiments were performed by an investigator blinded to mouse identity.

Immunoblots of sciatic nerve: Sciatic nerves were harvested 2 days after PNL to identify early molecular and cellular changes. Approximately 0.5 cm of sciatic nerve was collected distal from the ligation site. Ipsilateral and contralateral nerves were collected. Nerves were lysed in RIPA buffer and equal amounts of protein (20 μg) from nerves lysates, as determined by BCA Protein Assay (Bio-Rad, Hercules, CA, USA), were subjected to 10% SDS-PAGE and electro-transferred to nitrocellulose membranes. The membranes were blocked with 5% nonfat dried milk and then incubated with anti-TLR4(CD284)/MD2 (BioLegend, San Diego, CA, USA; cat #117601, 1:1000), anti-CD11b (Abcam, Cambridge, MA, USA, cat #Ab1333357) and anti-β-actin (Cell Signaling Technology, Danvers, MA, USA; cat #1:1000). Primary antibodies were detected with HRP-conjugated species-specific secondary antibodies (Cell Signaling Technology, Danvers, MA, USA; cat #7076S or 4S; 1:5000). Immunoblots were developed using the SuperSignal West Pico PLUS chemiluminescent substrate (Thermo Fisher Scientific, Waltham, MA, USA), and the Protec Ecomax X-ray film processor. Densitometry analysis was performed using the Image J software (U. S. National Institutes of Health, Bethesda, MD, USA).

Immunohistochemistry of DRGs: DRGs were embedded in paraffin. For IHC studies, 4 μm thick DRG tissue sections were immunostained for CD11b (Abcam, Cambridge, MA, USA, cat #Ab1333357; 1:4500) or GFAP (Dako, Santa Clara, CA, USA; cat #$Z0334; 1:4000). Slides were immunostained using a Ventana Discovery Ultra (Ventana Medical Systems, Oro, AZ, USA). Antigen retrieval was performed using CC1 (tris-based; pH 8.5) for 40 min at 95° C. The primary antibodies CD11b and GFAP were incubated with the slides for 32 min at 37° C. The secondary antibody, OmniMap anti-HRP (Ventana Medical Systems, Oro, AZ, USA; cat #760-4311), was incubated on the sections for 12 min at 37° C. Antibodies were visualized using diaminobenzidine as a chromogen followed by hematoxylin as a counterstain. Slides were rinsed, dehydrated through alcohol and xylene and cover slipped. Light microscopy was performed using a Leica DFC420 microscope with Leica Imaging Software 2.8.1 (Leica Biosystems, Vista, CA, USA).

Statisticalanalysis: Statistical analysis was performed using GraphPad Prism (GraphPad Prism version 9.1.2 for Mac, GraphPad Software, San Diego, CA, USA). All results are expressed as the mean±SEM. Comparisons between two groups were performed using two-tailed unpaired T-tests. A non-parametric Mann-Whitney U test was used when the variance in the two populations were significantly different. When greater than two groups were compared, a one-way ANOVA and Tukey's post hoc test was performed or in the case of non-parametric data, the Kruskal-Wallis test was utilized. Measurements of neuropathic pain, in which multiple observations in individual mice over time, were collected and analyzed by repeated-measures ANOVA with a Sidak's post hoc test. p<0.05 was considered statistically significant.

### Example 1: Identification of Anti-Inflammatory Motif

As demonstrated herein, a small peptide fragment of the C-terminal end of alpha-1 antitrypsin (the prototypical SERPIN) is capable of binding to LRP1, exerting potent cell regenerative, tissue protective, and immune-modulatory functions. Interestingly, the naturally occurring degradative C-terminal product of Alpha-1 Anti-trypsin (termed C-36) exhibits pro-inflammatory activity similar to lipopolysaccharide in both macrophages and neutrophils.⁴⁴ By excising a short fragment of the C-terminal end of Alpha-1 anti-trypsin, the anti-inflammatory sequences were identified. The amino acid sequences of the peptides tested are shown in Table 1 below. The core sequences containing the LRP1 binding site for each peptide are shown in bold and underlined.

TABLE 1
Peptides Tested for Anti-Inflammatory Activity

| | SEQ | |
|---|---|---|
| | ID | |
| Peptides | NO: | Sequences |
| SP34 (Scrambled Core) | 22 | F P K M V P Q F N T E L K I F P E V N I K |
| SP8 (AAT C-36 Peptide) | 23 | S I P P E V K F N K P F V F L M I E Q N T |
| | | K S P L F M G K V V N P |
| SP16 (AAT Core) | 24 | V K F N K P F V F L M I E Q N T K |
| SP20 (SP16 Short Core) | 25 | V K F N K P F V F L M |
| SP21 (SP16 Core Poly-R) | 26 | R R R V K F N K P F V F L M I E Q N T K |
| | | R R R |
| SP22 (SP16 Short Core Poly-R) | 27 | R R R V K F N K P F V F L M R R R |
| SP23 (SERPIN 2 Core) | 28 | L R F N R P F L V V I F S T S T Q |
| SP24 (SERPIN 2 Short Core) | 29 | L R F N R P F L V V I |
| SP26 (SERPIN 2 Short Core Poly-R) | 30 | R R R L R F N R P F L V V I R R R |
| SP28 (SERPIN 3 Short Core) | 31 | V R F N R P F L M I I |
| SP29 (SERPIN 3 Short Core Poly-R) | 32 | R R R V R F N R P F L M I I R R R |
| SP31 (SERPIN No LRP1 Site, which | 33 | V R F N R P F L |
| has the LRP1 binding site truncated) | | |
| SP32 (SERPIN No LRP1 Site Poly-R) | 34 | R R R V R F N R P F L R R R |

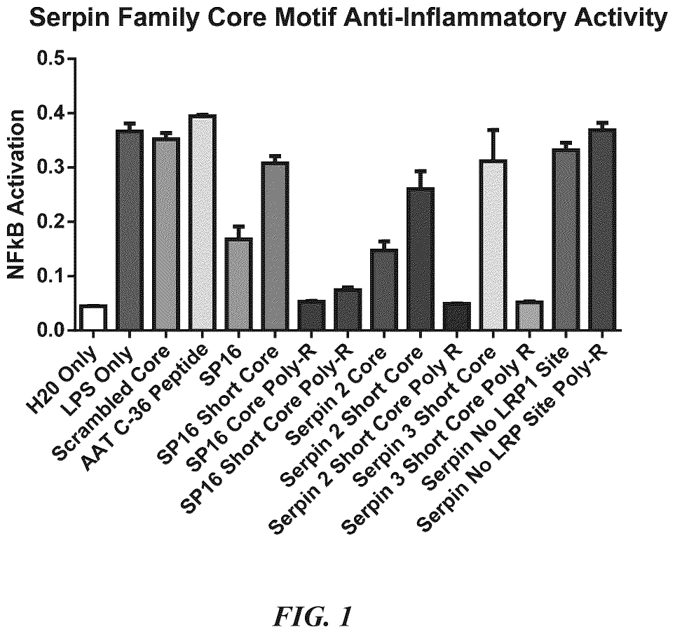

The reporter cells (THP1-XBlue-MD2-CD14 cells) were treated with each peptide (50 μg/ml) before being insulted with LPS (5 ng/ml) and incubated overnight. The NFκB inducible Secreted Embryonic Alkaline Phosphatase (SEAP) was measured in the supernatant and read for absorbance. As shown in FIG. 1, various SERPIN peptides sharing a common core motif demonstrated anti-inflammatory activity in NFκB reporter cells when insulted with LPS. LPS insult led to an increase in NFκB activity, and neither the scrambled core control peptide nor the AAT 0-36 fragment decreased the NFκB activity. In fact, the AAT 0-36 fragment showed NFkB inducible properties without the need for LPS.

In contrast, SP16 is capable of reducing NFκB activity, however, the short core peptide SP20 did not have an inhibitory effect. Although this truncated AAT derived peptide contains the core sequence and LRP1 binding site, it is unstable, and therefore exhibited no activity. When SP16 was flanked with triple arginine amino acids on both sides (termed “Poly-R”) to obtain SP21, the SP21 peptide's stability increased and NFkB inhibition increased as well relative to SP16. When the SP16 short core, SP20 peptide, was stabilized using triple arginine flanks to obtain SP22, the NFκB activity was significantly reduced. Similar effects were observed for the other two pairs of SERPIN peptides, SERPIN 2 short core (SP24) vs. SERPIN 2 short core poly-R (SP26), and SERPIN 3 short core (SP28) vs. SERPIN 3 short core Poly-R (SP29). Accordingly, this example demonstrates that shortening the peptide resulted in instability and lack of function, while stabilizing with the poly-R flanks resulted in heightened activity.

Moreover, these peptides all contain an LRP1 binding site, however, when the LRP1 binding site was truncated to obtain SP31, the anti-inflammatory activity of the peptide was lost and could not be restored with the poly arginine flanks (SP32 peptide). This indicates that SERPINs contain an anti-inflammatory core motif that is dependent on LRP1.

### Example 2: Primary Adult Sensory Neurons Sprout in Response to SP163M

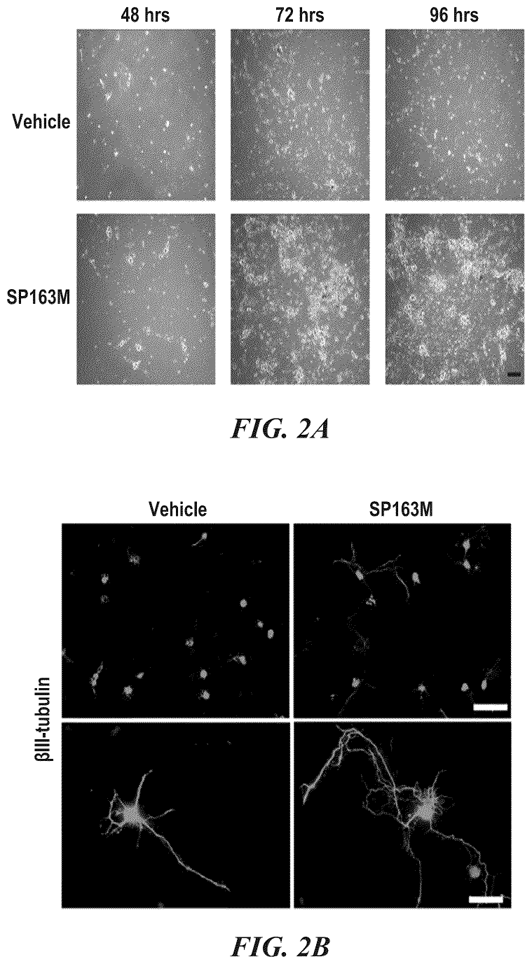

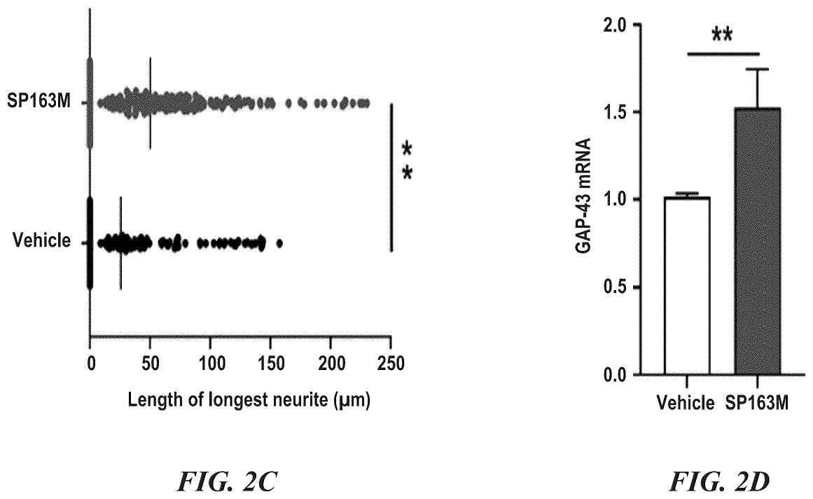

To determine whether SP163M possessed bioactivity in sensory neurons, primary adult rat DRG neurons were treated with SP163M for up to 96 h. Within 48 h, phase contrast images of DRG neuronal cultures revealed that SP163M induced greater neuronal survival and neurite sprouting compared to untreated controls (FIG. 2A). After 72 and 96 h in culture, the neuronal networks became extensive and continued to show a greater survival of neuronal cell bodies and neurite extensions.

To specifically identify neurons, immunofluorescence studies were performed with βIII-tubulin. Primary adult DRG neuron cultured on poly-L-lysine (PLL) and laminin showed basal levels of sprouting when treated with vehicle after 54 h (FIG. 2B). In contrast, cultured DRG neurons treated with SP163M showed significant levels of sprouting that included increases in both branching and length after 54 h. Quantification of the longest neurite indicated that SP163M was significantly neurotrophic (FIG. 2C). RT-qPCR analysis of a regenerative associated gene, growth associated protein, GAP-43, demonstrated that SP16 increased GAP-43 mRNA compared to vehicle-treated neurons (FIG. 2D).

### Example 3: SP163M Activates LRP1 Dependent Cell Signaling

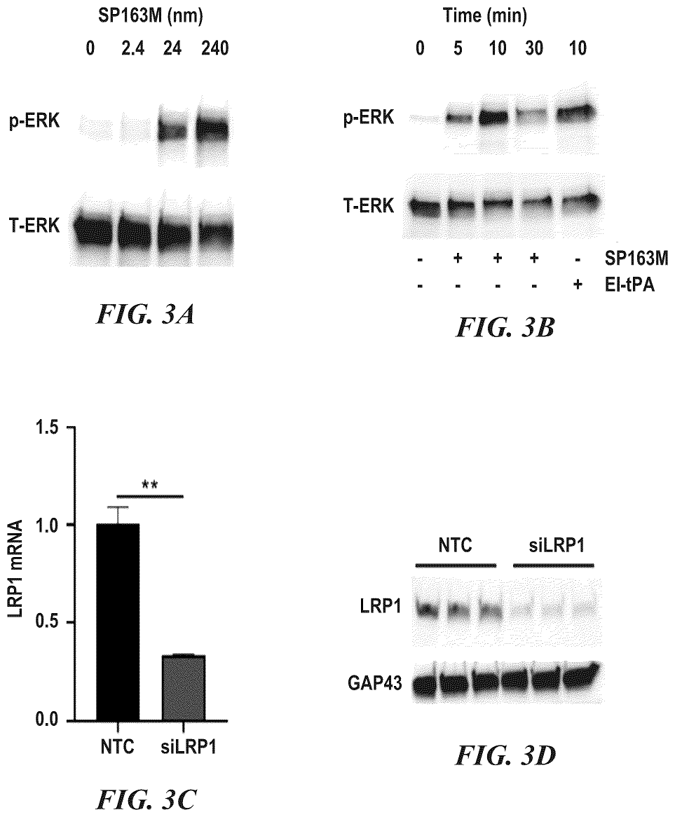

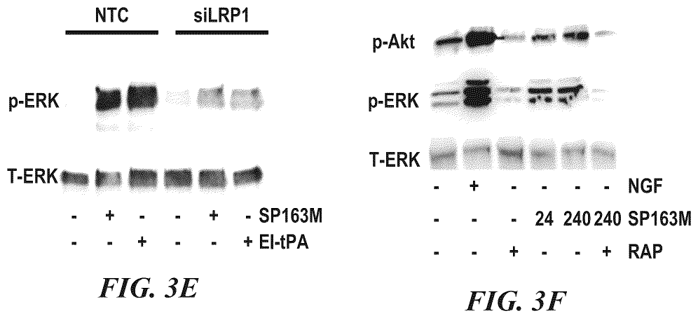

Previously, it was shown that LRP1 ligands, such as EI-tPA, robustly activated cell survival signaling in neurons and SCs, however, the effects of SP163M were unknown. To begin, several concentrations of SP163M was added to cultured PC12 cells for 10 min. SP16 activated phospho-ERK1/2 at concentrations as low as 24 nM (FIG. 3A). Next, SP16 was added to PC12 cells overtime. SP16 robustly activated ERK1/2 from 5 to 30 min (FIG. 3B). SP16 activation of ERK1/2 at 10 min was similar to the known LRP1 interactor, EI-tPA. EI-tPA is a derivative of tissue-type plasminogen activator (tPA), a protease that is an activator of fibrinolysis, and is a globally approved drug for treating non-hemorrhagic stroke. Next, LRP1 expression was silenced with siRNA (siLRP1). PC12 cells transfected with siLRP1 showed significantly decreased (70%) expression of LRP1 mRNA compared with cells transfected with NTC siRNA for 48 h (FIG. 3C). LRP1 protein also was significantly reduced after 48 h (FIG. 3D). When PC12 cells transfected with NTC siRNA were treated with SP16, ERK1/2 was robustly activated. EI-tPA also robustly activated ERK1/2, as anticipated. In contrast, ERK1/2 was not activated by SP163M or EI-tPA in cells transfected with LRP1-specific siRNA (FIG. 3E). SP163M also activated Akt and EKR1/2 in PC12 cells after 10 min that was blocked by the addition of the 39 kDa LRP1 antagonist, RAP (150 nM), as expected for any LRP1-dependent agonist (FIG. 3F). NGF also activated ERK1/2, as anticipated, and served as a cell signaling control. Collectively, these data support that SP163M is bioactive in sensory neurons and sensory-like neurons via an LRP1 dependent pathway.

### Example 4: SP163M Modulates Primary Afferent Input and Reduces Central Sensitization in the Later Phase of the Formalin Test

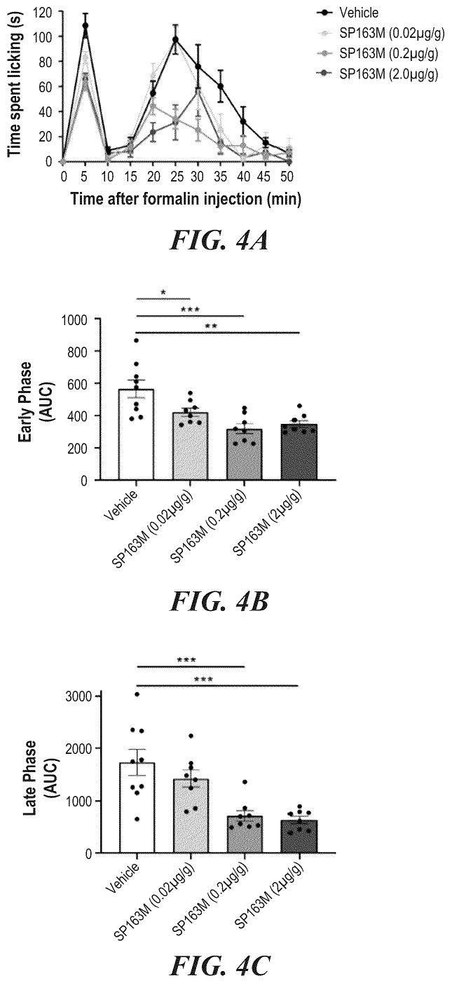

The formalin test is a tissue injury model with an acute nociceptive first phase and an inflammatory second phase.⁶² It is a widely used tool to screen analgesic and anti-inflammatory pain therapeutics. It was tested whether LRP1 agonism by SP163M regulated pain responses induced by intraplantar formalin (FIG. 4A). SP163M (0.02, 0.2, or 2.0 μg/g) or vehicle was administered one hour prior to paw injection of 2.5% formalin solution. Preemptive administration was initially studied to understand the contribution of primary afferent input in the early phase and is an approach consistent with other formalin test studies testing initial effects of novel opioids or other analgesics.^{62,63} Time spent licking was quantified by two blinded observers over 50 min. Vehicle treated mice showed the characteristic pattern of paw licking during the two phases. In the early phase (0-10 min), vehicle-treated mice robustly licked their injected hind paw for over 100 s. However, SP163M treated-mice demonstrated dose-dependent reductions in licking time (0.02 μg/g; 80 s) and high (2 μg/g; 60 s). Analysis of area under the curve (AUC) revealed that both the low and high SP163M treatment groups reached statistical significance (FIG. 4B). In the later phase (15-50 min), vehicle-treated mice showed peaked paw licking (90 s) after 25 min that resolved at 50 min, as anticipated (FIG. 4A). In contrast, the highest dose of SP163M (2 μg/g) delayed the peak late phase start time to 30 min. Analysis of AUC revealed that both middle (0.2 μg/g) and high (2 μg/g) doses reduced licking time in the late phase compared to vehicle controls (FIG. 4C). These findings indicate that LRP1 activation by SP16 reduced both the nociceptive and ongoing sensitization/inflammatory phase of the formalin test.

### Example 5: LRP1 Ligands Block Acute Nociception

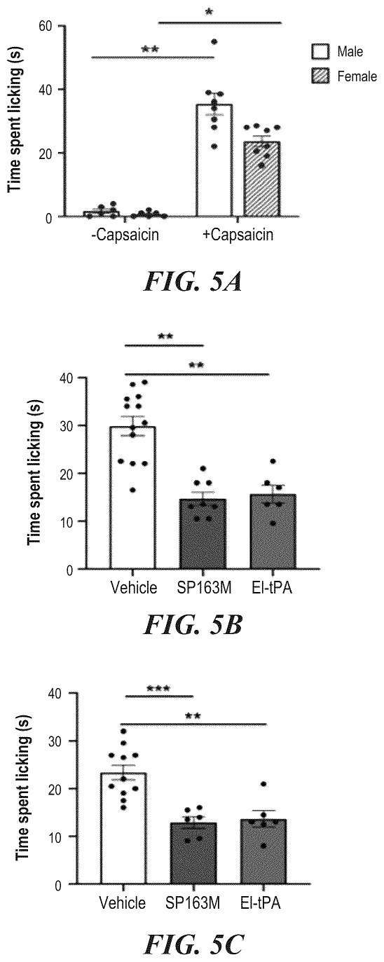

Intraplantar injection of capsaicin, the lipophilic vanilloid compound found in “hot” chili peppers⁶⁴ binds and activates the transient receptor potential vanilloid 1 (TRPV1) in nociceptive peripheral terminals.⁶⁵ This induces ion influx and action potential firing associated with burning pain resulting in a licking response toward the injected paw. The acute spontaneous pain-related behaviors induced by capsaicin are transient (<10 min) with most of the activity occurring during the first 2-3 min. Initially, it was confirmed that capsaicin increased pain-related behaviors in male and female mice when using 20% cyclodextrin as the vehicle for the capsaicin solution. Both male and female mice manifested a greater licking response to capsaicin injection than vehicle injection; the males tended to be slightly more sensitive (FIG. 5A).

Because anti-nociceptive activities have not been previously shown for any LRP1 interactor, both EI-tPA and SP163M were tested. EI-tPA or SP16 was delivered systemically and one hour preemptively prior to capsaicin injection in both male and female mice. In male mice, SP163M and EI-tPA, blocked the capsaicin induced acute pain-related behavior (FIG. 5B). Similar effects of LRP1 agonists were observed in female mice (FIG. 5C).

### Example 6: SP163M Inhibits the Development of Neuropathic Pain, Suppresses Innate Immunity and Reduces Inflammatory Cell Recruitment

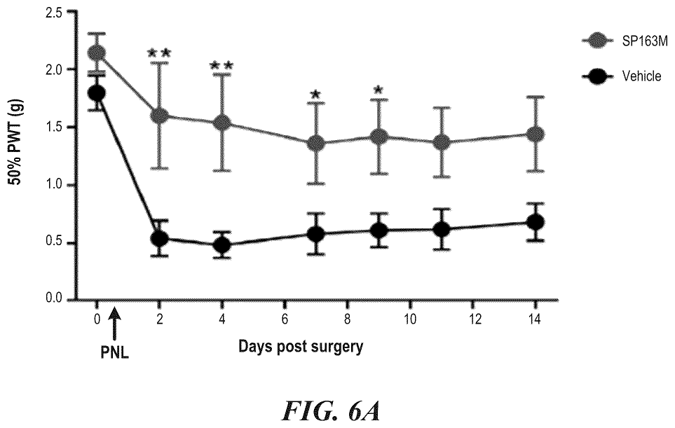

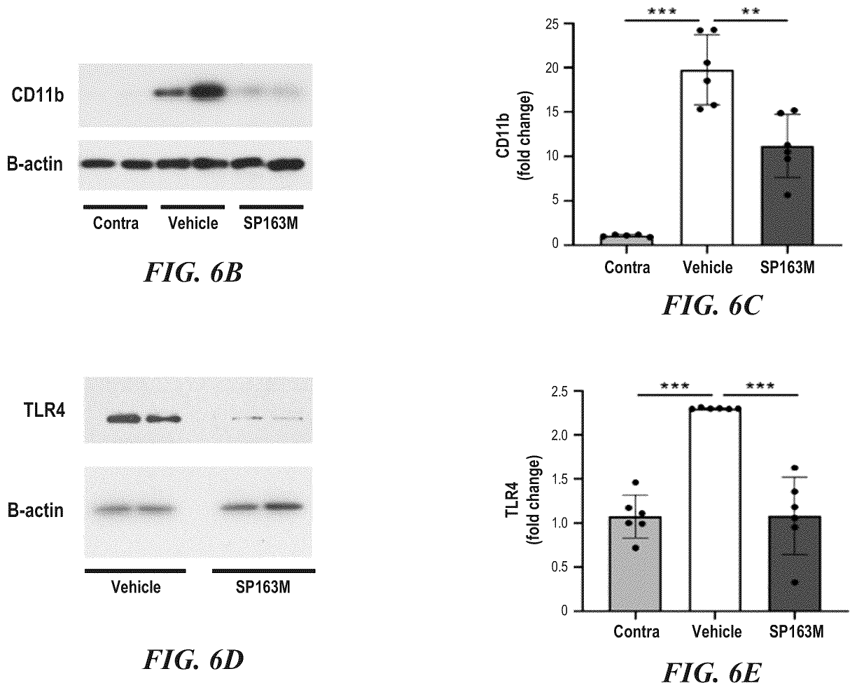

Next, SP163M was tested in a neuropathic pain model, PNL. PNL in the sciatic nerve induces mechanical hypersensitivity that can be observed within two days after injury.⁵⁷ Male mice were randomly placed into groups, baselined with von Frey filaments and paw withdrawal thresholds (PWT) were recorded over one week prior to PNL. SP163M (2 μg/g) was given preemptively one hour prior to PNL and then daily for two weeks to test whether SP163M was neuroprotective. Vehicle-treated mice developed tactile allodynia by day 2 and hypersensitivity continued for 2 weeks (FIG. 6A), as anticipated. In contrast, mice that were treated with SP163M did not develop mechanical hypersensitivity. Statistically, the effects of SP163M were most pronounced early after injury (day 2 and 4), nonetheless, the anti-allodynic effects of SP163M were sustained throughout day 9 post injury. These results indicate that activation of LRP1 may prevent the development of mechanical sensitivity induced by direct nerve damage.

It was reported that LRP1 induces potent anti-inflammatory activity in macrophages, during myocardial infarction, and can regulate innate immunity. To determine whether SP163M modulates neuroinflammation in the injured PNS, sciatic nerves were collected from vehicle and SP163M treated groups two days after PNL. CD11b was used to identify inflammatory cells present in the nerve. CD11b was robustly increased in nerves immediately distal to the ligation site in vehicle treated mice, as anticipated (FIG. 6B). In contrast CD11b was decreased in nerves treated with SP163M. Densitometric analysis revealed that CD11b was increased almost 20-fold (FIG. 6C, p<0.001) and SP163M reduced the levels of CD11b by 10-fold (p<0.01). Because activation of TLR4 is associated with pain states, TLR4 in injured sciatic nerves was also measured. Immunoblots revealed that TLR4 was upregulated in nerve two days after PNL and that SP16 robustly down regulated TLR4 expression (FIG. 6D). Densitometric analysis revealed that TLR4 increased greater than 2-fold after injury in vehicle treated mice and that SP163M completely blocked the upregulation of TLR4 (FIG. 6E).

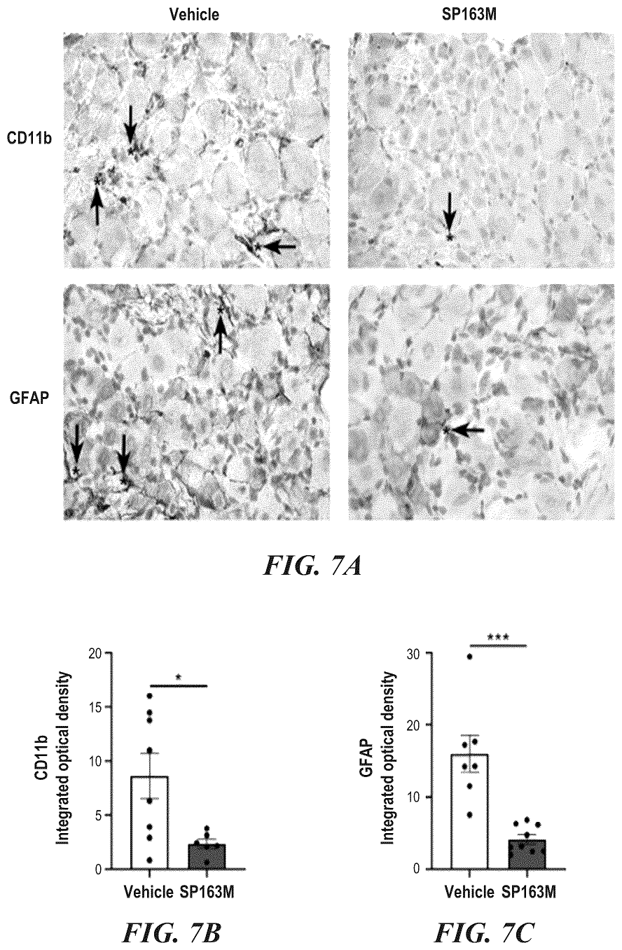

Recently, it has been shown that inflammatory cells and macrophages infiltrating into the DRGs acutely after nerve injury directly regulate chronic pain states. Accordingly, L4 DRGs were collected from mice that received vehicle or SP163M treatment two days after PNL. Immunohistochemistry was performed on transverse DRG sections to identify CD11b (FIG. 7A). In PNL sections, CD11b immunoreactivity was observed in between neuronal cell bodies and around blood vessels.

In contrast, transverse sections of SP163M treated nerves revealed little immunoreactivity, indicating a very low level of inflammatory cells present. Quantification of CD11B immunohistochemistry showed that SP163M treated DRGs had approximately five-fold less CD11b levels than vehicle-treated DRGs (FIG. 7B). Next, satellite cell activation was examined. Satellite cells in naïve DRGs do not express GFAP, however after injury, satellite cells abundantly express GFAP. Two days after PNL, satellite cells showed robust GFAP immunoreactivity in vehicle treated DRGs. Conversely, mice that were treated with SP163M showed significantly less GFAP expression in DRGs. Quantification of GFAP immunohistochemistry revealed a 6-fold decrease in GFAP levels in SP163M treated mice (FIG. 7C). These findings suggest that engagement of LRP1 reduces satellite cell activation.

Accordingly, Examples 2-6 demonstrate the robust efficacy of SP163M in three distinct pre-clinical mouse models that includes acute nociceptive, inflammatory, and neuropathic pain. Central to the effect of preventing mechanical hypersensitivity by SP163M, was its potent anti-inflammatory activity in injured peripheral nerves. In these studies, SP163M robustly reduced the early recruitment of inflammatory cells distal to the nerve injury site and in the corresponding L3, L4 DRG early after sciatic nerve ligation. SP163M can delay and/or limit the infiltration of inflammatory cells and thereby regulate pain states.

### Example 7: SP163M Blocks IL-13 Stimulated Stat6 Phosphorylation

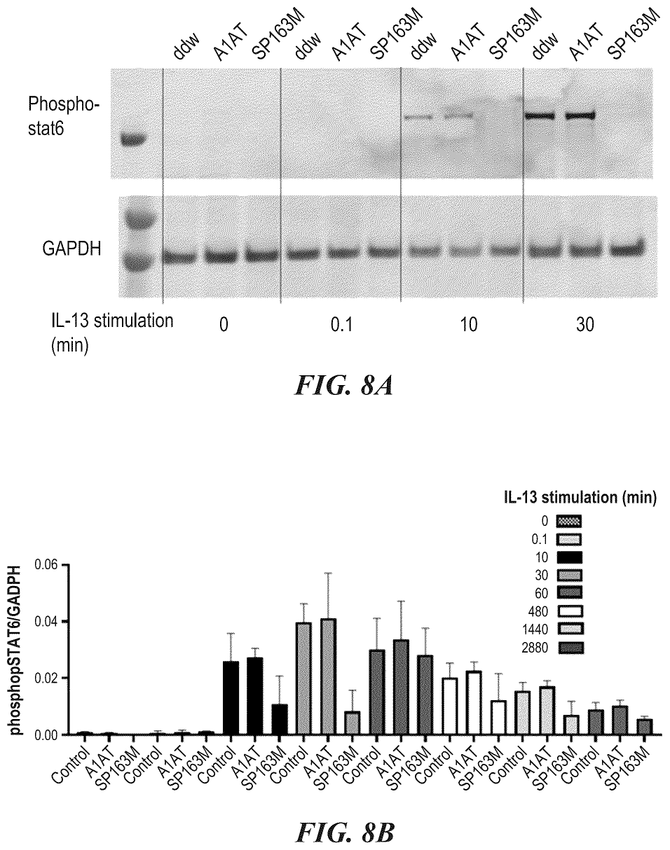

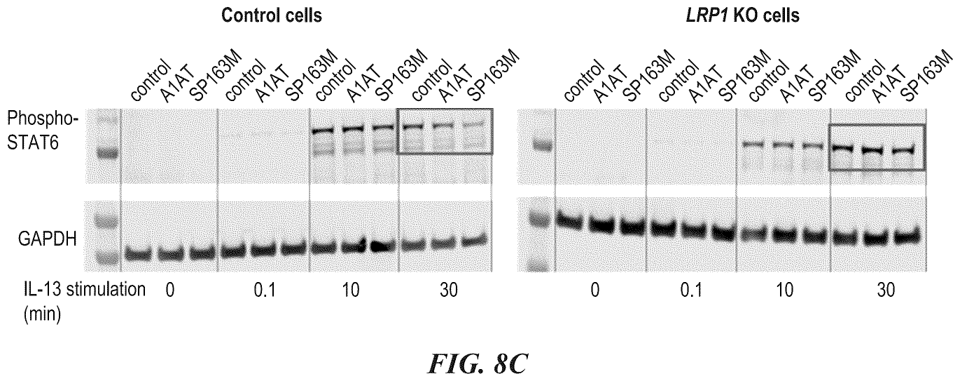

Esophageal EPC2 cells were treated with vehicle (ddw), A1AT or SP163M. Cells were collected at time-points indicated and western blot analysis of phospho-specific STAT6 was analyzed. At 30 minutes post IL-13 induction, STAT6 was phosphorylated in vehicle and A1AT treated cells but not in SP163M treated cells (FIG. 8A). This reduction in STAT6 phosphorylation by SP163M persisted for several hours post-treatment (many different experimental replicates shown) (FIG. 8B). FIG. 8C shows that the SP163M reduction of phosphorylated STAT6 was dependent on expression of LRP1. Using CRISPER/CAS9 technology, an LRP1 knockout esophageal EPC2 cell line was created. In the control cells (with LRP1) SP163M was capable of reducing phospho-STAT6 expression; however, in the LRP1 knockout cell line, SP163M was not capable of reducing phosphorylated STAT6.

### Example 8: SP163M Shows Inhibitory Effect in an Additional Model of Eosinophilic Esophagitis Using A. alternata as Allergen

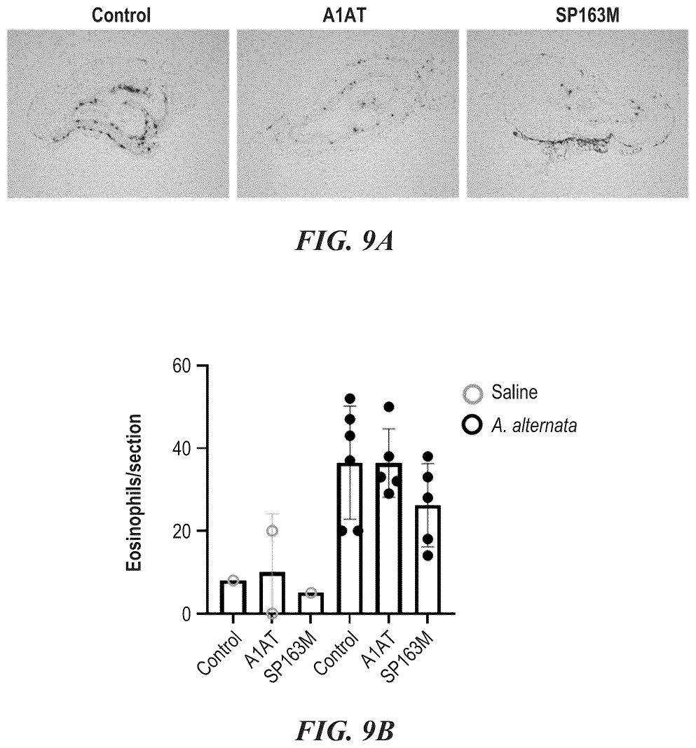

Balb/C mice were repeatedly challenged with a series of the allergen A. alternata over 4 weeks. A. alternata is a common airborne mold associated with eosinophilic inflammation and a variety of allergic diseases, such as rhinitis, asthma and dermatitis, and is an established model that induces allergic reaction at the esophagus. In this model, the allergen challenges lead to an increase in esophageal eosinophilia. SP163M, A1AT or vehicle (control) was given twice weekly over the 4-week period for 8 total treatments. At the end of the study, esophageal sections were stained for the detection of MBP (eosinophilic marker) and quantitated per high power field. Treatment with SP163M decreased the number of eosinophils that infiltrated to the esophagus compared to both control mice which were treated with vehicle as a control and mice treated with A1AT at a higher dose than SP163M (FIG. 9).

### Example 9: SP163M Demonstrates Effects on Atopic Dermatitis

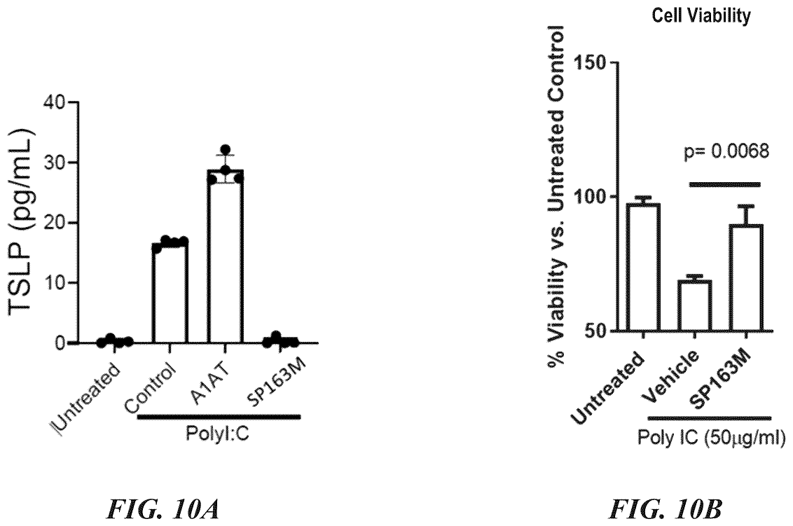

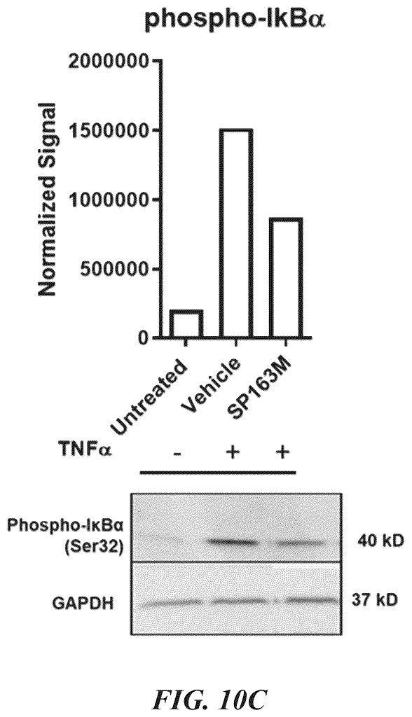

Like human esophageal epithelial cells, human keratinocytes treated with Poly I:C resulted in an increase in TSLP production and an associated increase in cell death. SP163M treatment of human keratinocytes induced with poly I:C resulted in a decrease of both TSLP (FIG. 10A) and an increase in cell viability (FIG. 10B). In this model of dermal inflammation, SP163M decreased a key cytokine (TSLP) that controls allergic responses, and protected cells from death.

In epidermal keratinocytes (HaCat) cells, SP163M treatment inhibited the phosphorylation of IκBα by TNF-α, an inflammatory cytokine in an in vitro model of atopic dermatitis. TNFα treatment led to phosphorylation of IκBα (inhibitor of nuclear factor kappa B), which then activated NFκB. Therefore, in a model of skin inflammation, SP163M shut down a key inflammatory pathway (FIG. 10C).

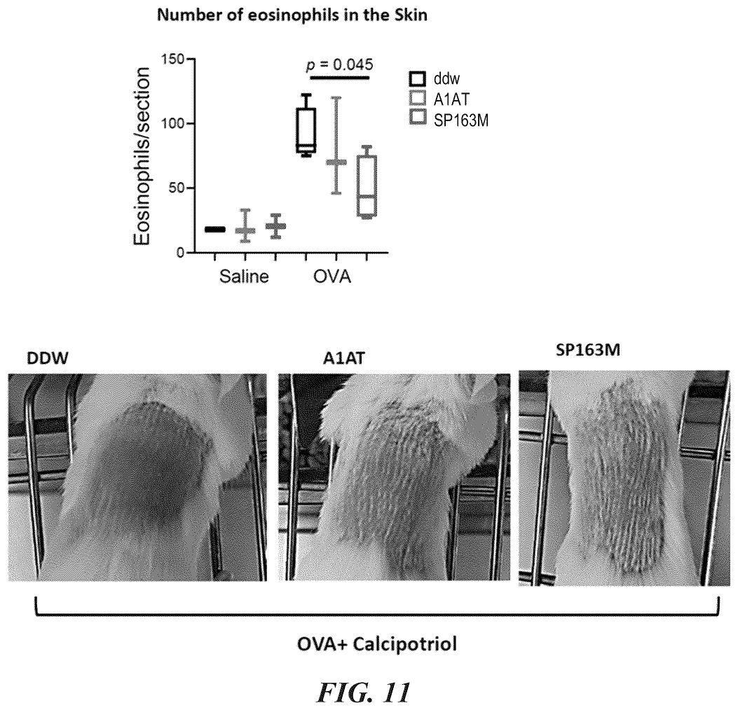

In a model of calcipotriol/ova induced atopic dermatitis in mice, treatment with SP163M led to significantly lower levels of eosinophils per section compared to vehicle control (p=0.045). SP163M treatment led to decreased eosinophilic infiltration compared to A1AT given at 20× higher dose (FIG. 11). Accordingly, SP163M improved outcomes in the atopic dermatitis animal model.

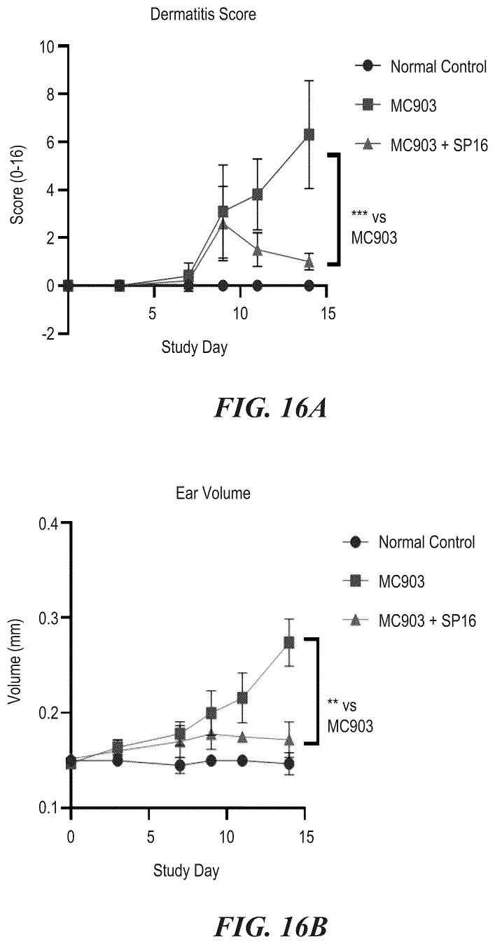

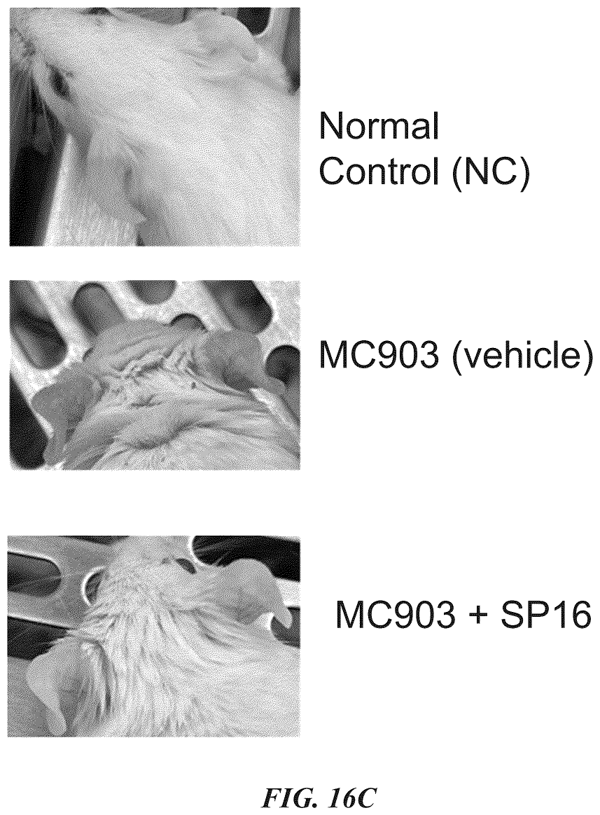

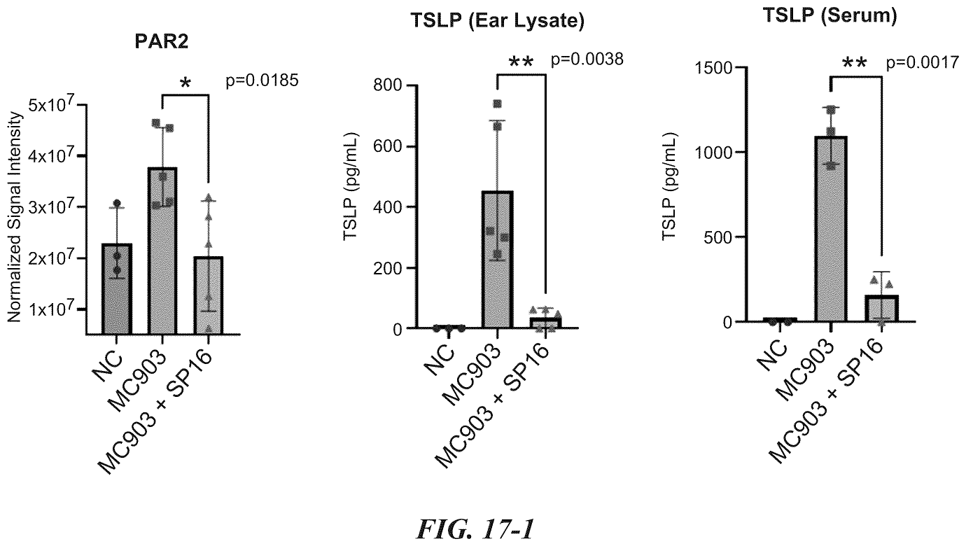

As shown in FIG. 16A-16C, in an atopic dermatitis model induced by calcipotriol over a 14-day period, mice treated with SP16 topical solution (1%) shows a significant improvement in disease vs. vehicle-treated mice.

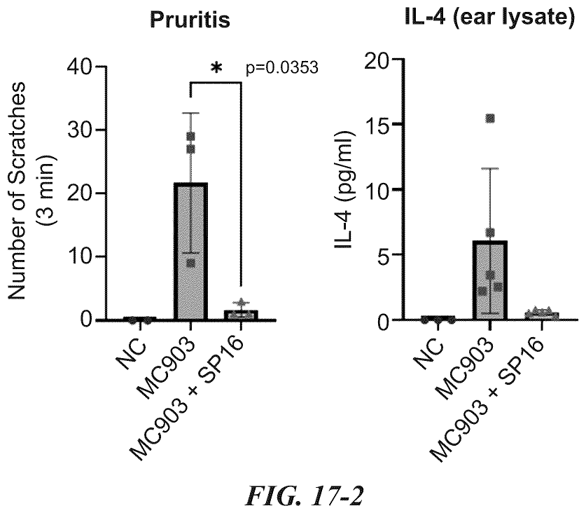

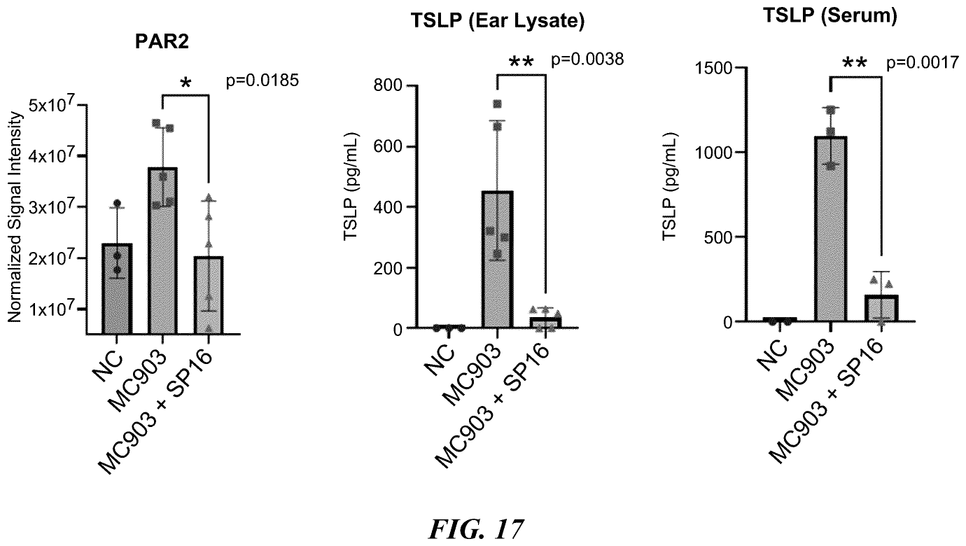

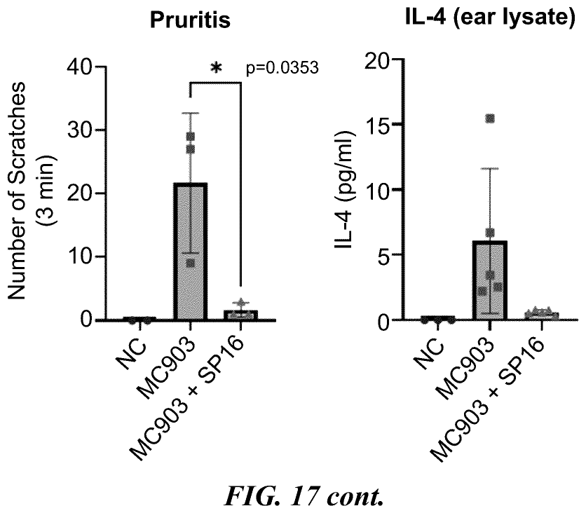

As shown in FIG. 17, PAR2 and TSLP are upregulated in skin biopsies of AD mice. Inhibition of these upstream mediators results in attenuated scratching and inflammation in mice. SP16 treated mice show a significant reduction in systemic (serum) and local (ear) TSLP compared to MC903 treated mice. SP16 treatment significantly reduces pruritis compared to vehicle (MC903) treated mice. Downstream TH2-mediated IL-4 release in the skin tissue is significantly reduced in SP16-treated animals.

### Example 10: SP16 Inhibits Key Allergic Inflammatory Mediators

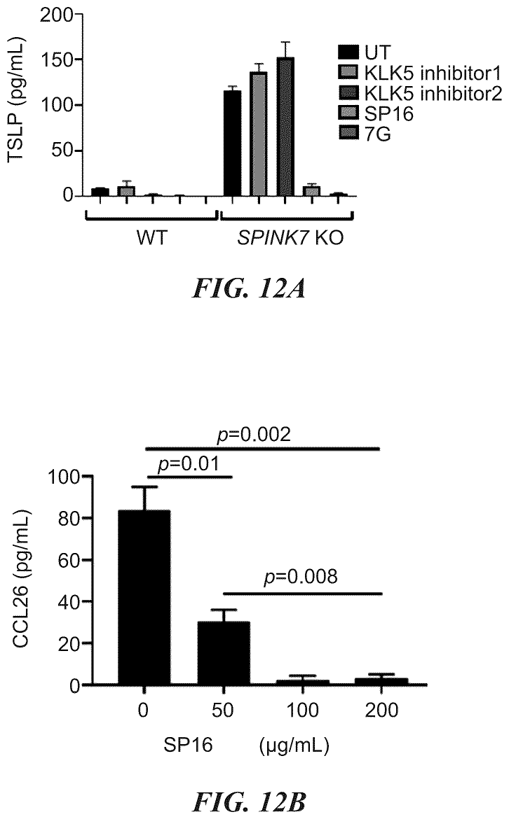

SP16 reduces several key cytokines involved in allergic responses. Upon knockout of SPINK7, esophageal epithelial cells release exacerbated levels of TSLP. TSLP is a key mediator of immune cell responses upon activation to an insult such as allergens. Likewise, CCL26 is a chemokine involved in allergen induced eosinophilic activation, also upregulated upon SPINK7 knockout. In primary SPINK7 knockout esophageal cells, SP16 (and an analog of SP16 that targets LRP1, 7G) are effective in significantly reducing Poly I:C mediated TSLP and CCL26 release (FIG. 12).

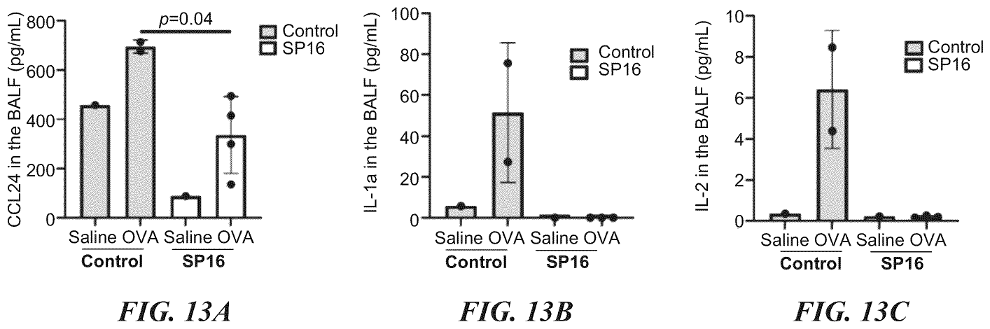

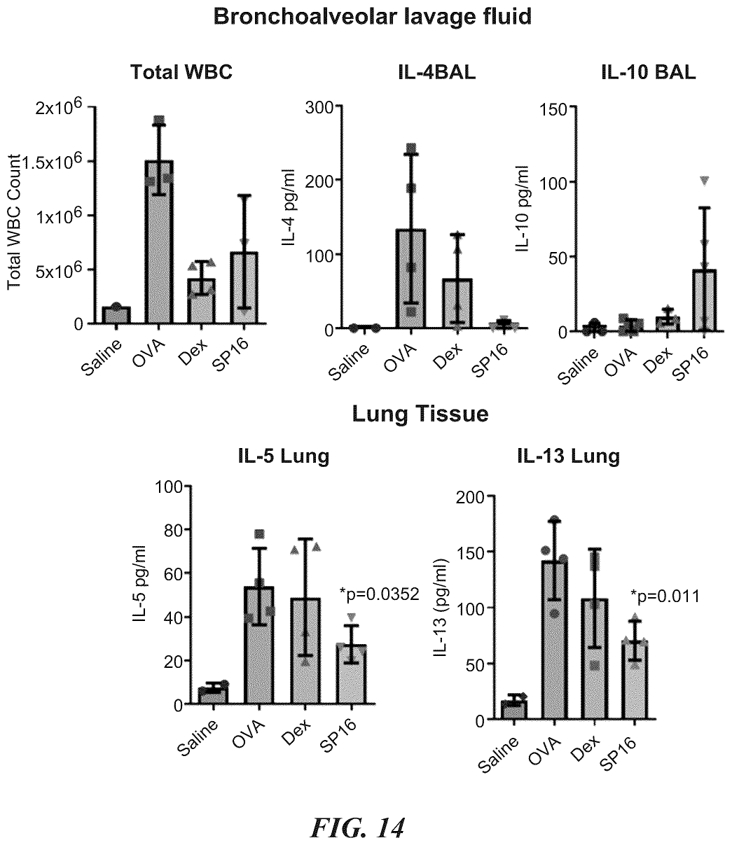

The ova-induced model of allergic inflammation is a widely used model of eosinophilic driven allergic diseases. As shown in FIGS. 13-14, using the ova-induced allergic model in mice, SP16 significantly reduces cytokines in the bronchoalveolar lavage fluid (BALF).

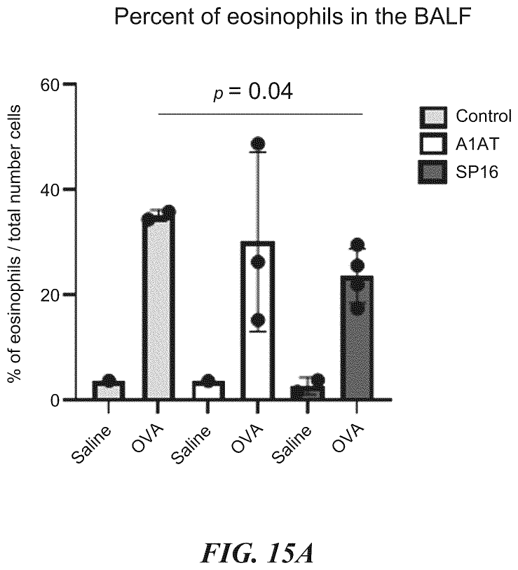

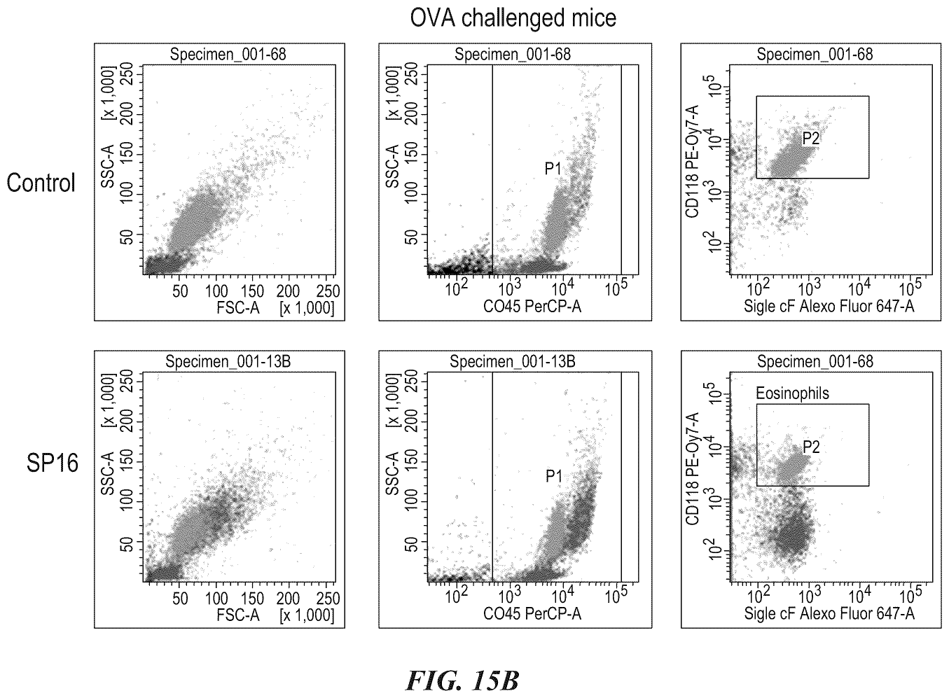

The impact of SP16 treatment on eosinophilic infiltration in mice was assessed using the OVA-induced allergic inflammation model. The number of eosinophils in the bronchoalveolar lavage fluid was quantified by flow cytometry. FIG. 15 shows SP16 significantly (p=0.04) reduces eosinophil infiltration into the lungs.

### Additional Examples

Uses for SERPIN Peptides in Reducing Inflammation in a Subject Having a Disease or Condition Associated with LRP1 or TSLP

In some embodiments, the technology includes use of a SERPIN peptide in reducing inflammation in a subject having a disease or condition associated with LRP1 or TSLP. In some aspects, the technology includes administering any SERPIN peptide described in this disclosure.

In some aspects, use of the SERPIN peptide in reducing inflammation in a subject having a disease or condition associated with LRP1 or TSLP comprises administering a SERPIN peptide comprising an amino acid sequence selected from the group consisting of VKFNKPFVFL(NIe)IEQNTK (SEQ ID NO: 35), VKFNKPFVFLM (SEQ ID NO: 25), VKFNKPFVFLMIEQNTK (SEQ ID NO: 2), LRFNRPFLVVI (SEQ ID NO: 29), VRFNRPFLMII (SEQ ID NO: 31), VKFNKPFVFL(NIe) (SEQ ID NO: 40), RFNRPFLVVIR (SEQ ID NO: 41), RFNRPFLMIIR (SEQ ID NO: 42), RFNKPFVFL(NIe)R (SEQ ID NO: 43), RRRFLVVIRRR (SEQ ID NO: 44), RRRFLMIIRRR (SEQ ID NO: 45), RRRFVFL(NIe)RRR (SEQ ID NO: 46), FVFLM (SEQ ID NO: 3), and FVFL(NIe) (SEQ ID NO: 10), wherein the SERPIN peptide is administered to the subject to reduce inflammation associated with the disease or condition associated with LRP1 or TSLP.

In some aspects, use of the SERPIN peptide in reducing inflammation in a subject having a disease or condition associated with LRP1 or TSLP comprises administering a SERPIN peptide comprising the SP16 peptide (SEQ ID NO: 2) or the SP163M peptide (SEQ ID NO: 35). In some aspects, the SERPIN peptide comprises an amino acid sequence that shares at least 95% identity with the SP16 peptide (SEQ ID NO: 2). In some aspects, the SERPIN peptide comprises an amino acid sequence that shares at least 95% identity with the SP163M peptide (SEQ ID NO: 35). In some aspects, the SERPIN peptide comprises an amino acid sequence that shares at least 90% identity with the SP16 peptide (SEQ ID NO: 2). In some aspects, the SERPIN peptide comprises an amino acid sequence that shares at least 90% identity with the SP163M peptide (SEQ ID NO: 35).

In some aspects, use of the SERPIN peptide in reducing inflammation in a subject having a disease or condition associated with LRP1 or TSLP comprises administering a SERPIN peptide comprising an amino acid sequence of X1-N-X2-P-F-X3-X4-X5-X6, wherein X1 is R or F, X2 is K or R, X3 is V or L, X4 is F, V, or M, X5 is L, V, or I, and X6 is M, I, or NIe. In some aspects, the SERPIN peptide comprises a sequence of FNKPFVFLM (SEQ ID NO: 1), FNKPFVFL[NIe] (SEQ ID NO: 5), FNRPFLVVI (SEQ ID NO: 6), FNRPFLVV[NIe] (SEQ ID NO: 7), FNRPFLMII (SEQ ID NO: 8), or FNRPFLVI[NIe] (SEQ ID NO: 9).

In some aspects, use of the SERPIN peptide in reducing inflammation in a subject having a disease or condition associated with LRP1 or TSLP comprises administering a SERPIN peptide comprising an amino acid sequence of X1-Z1-X2-Z2-X3-Z3-F-V-F-L-X4-Z4 (SEQ ID NO: 20), wherein: X1 is V or L; X2 is R or F; X3 is R or K; X4 is M, NIe, or I; Z1 is any amino acid; Z2 is any amino acid; Z3 is any amino acid; and Z4 is a sequence any five amino acids. In some aspects, the SERPIN peptide comprises of an amino acid sequence of X1-Z1-X2-Z2-X3-Z3-F-X4-F-L-Z4-X5 (SEQ ID NO: 21), wherein: X1 is V or L; X2 is F or R; X3 is K or R; X4 is V, L, or M; X5 is a sequence of any five amino acids; Z1 is any amino acid; Z2 is a sequence of any two amino acids; Z3 is any amino acid; and Z4 is M, NIe, or I.

In some aspects, use of the SERPIN peptide in reducing inflammation in a subject having a disease or condition associated with LRP1 or TSLP comprises administering a SERPIN peptide comprising the amino acid sequence of the SERPIN peptide comprising the sequence of SEQ ID NO: 35. In some aspects, the amino acid sequence of the SERPIN peptide comprises the sequence of SEQ ID NO: 2. In some aspects, the N-terminus of the SERPIN peptide is acetylated. In some aspects, the C-terminus of the SERPIN peptide is amidated. In some aspects, the SERPIN peptide is fused to one or more other peptides to form a fusion peptide or fusion protein. In some aspects, the one or more other peptides is different from the SERPIN peptide. In some aspects, the fusion peptide or fusion protein comprises the SERPIN peptide and an epitope tag, a half-life extender, or both the epitope tag and the half-life extender. In some aspects, the technology includes a pharmaceutical composition comprising the SERPIN peptide and a pharmaceutically effective carrier.

In some embodiments, use of the SERPIN peptide in reducing inflammation in a subject having a disease or condition associated with LRP1 or TSLP comprises administering a SERPIN peptide at a therapeutically effective dose or concentration. In some aspects, the pharmaceutical compound is administered at a therapeutically effective dose. In some aspects, the SERPIN peptide is administered at a clinically effective dose or concentration. In some aspects, the pharmaceutical compound is administered at a clinically effective dose. In some aspects, the SERPIN peptide is administered at a dose of between 0.001 mg/kg and 5 mg/kg. In some aspects, the subject is a human. In some aspects, the administration is by oral administration, parenteral administration, intradermal administration, transdermal administration, topical administration, or intranasal administration. In some aspects, the SERPIN peptide is administered as a single dose. In some aspects, the SERPIN peptide is administered by topical administration.

In some aspects, use of the SERPIN peptide in reducing inflammation in a subject having a disease or condition associated with LRP1 or TSLP comprises administering a SERPIN peptide, wherein the disease or condition is caused by A. alternata. In some aspects, the disease or condition is rhinitis, asthma, dermatitis, or esophageal eosinophilia. In some aspects, the disease or condition is rhinitis. In some aspects, the disease or condition is asthma. In some aspects, the disease or condition is dermatitis. In some aspects, the disease or condition is esophageal eosinophilia. In some aspects, the disease or condition is an acute or neuropathic pain. In some aspects, the disease or condition is acute nociceptive, inflammatory, or neuropathic pain. In certain embodiments, the disease or condition is an EDD. In some aspects, the disease or condition is EoE, eosinophilic asthma, atopic dermatitis, nasal polyps, chronic spontaneous urticaria, and pruritis. In some aspects, the disease or condition is atopic dermatitis. In some aspects, the disease or condition is pruritis. In some aspects, the disease or condition is an allergic reaction. In some aspects, the disease or condition is allergic inflammation. In some aspects, the disease or condition is eosinophilic allergic inflammation. In some aspects, the disease or condition is caused by TH2 driven inflammatory cytokines.

### Uses for SERPIN Peptide in Treating Acute or Neuropathic Pain, Nociceptive Pain, or Inflammatory Pain

In some embodiments, the present technology includes use of a SERPIN peptide in the treatment of a disease associated with LRP1 or TSLP, where the disease or condition is acute or neuropathic pain, nociceptive pain, or inflammatory pain. In aspects, the technology includes use of any of the SERPIN peptides described in this application for the treatment of acute or neuropathic pain, nociceptive pain, or inflammatory pain.

In some aspects, the use of the SERPIN peptide in treating acute or neuropathic pain, nociceptive pain, or inflammatory pain comprises administering a SERPIN peptide comprising an amino acid sequence selected from the group consisting of VKFNKPFVFL(NIe)IEQNTK (SEQ ID NO: 35), VKFNKPFVFLMIEQNTK (SEQ ID NO: 2), VKFNKPFVFLM (SEQ ID NO: 25), LRFNRPFLVVI (SEQ ID NO: 29), VRFNRPFLMII (SEQ ID NO: 31), VKFNKPFVFL(NIe) (SEQ ID NO: 40), RFNRPFLVVIR (SEQ ID NO: 41), RFNRPFLMIIR (SEQ ID NO: 42), RFNKPFVFL(NIe)R (SEQ ID NO: 43), RRRFLVVIRRR (SEQ ID NO: 44), RRRFLMIIRRR (SEQ ID NO: 45), RRRFVFL(NIe)RRR (SEQ ID NO: 46), FVFLM (SEQ ID NO: 3), and FVFL(NIe) (SEQ ID NO: 10), wherein the SERPIN peptide is administered to a subject in need thereof to treat the disease or condition associated with LRP1 or TSLP, and wherein the disease or condition is acute or neuropathic pain, nociceptive pain, or inflammatory pain.

In some aspects, the use of the SERPIN peptide in treating acute or neuropathic pain, nociceptive pain, or inflammatory pain comprises administering a SERPIN peptide comprising SP16 peptide (SEQ ID NO: 2) or the SP163M peptide (SEQ ID NO: 35). In some aspects, the SERPIN peptide comprises an amino acid sequence that shares at least 95% identity with the SP16 peptide (SEQ ID NO: 2). In some aspects, the SERPIN peptide comprises an amino acid sequence that shares at least 95% identity with the SP163M peptide (SEQ ID NO: 35). In some aspects, the SERPIN peptide comprises an amino acid sequence that shares at least 90% identity with the SP16 peptide (SEQ ID NO: 2). In some aspects, the SERPIN peptide comprises an amino acid sequence that shares at least 90% identity with the SP163M peptide (SEQ ID NO: 35).

In some aspects, the use of the SERPIN peptide in treating acute or neuropathic pain, nociceptive pain, or inflammatory pain comprises administering a SERPIN peptide comprising an amino acid sequence of X1-N-X2-P-F-X3-X4-X5-X6, wherein X1 is R or F, X2 is K or R, X3 is V or L, X4 is F, V, or M, X5 is L, V, or I, and X6 is M, I, or NIe. In some aspects, the SERPIN peptide comprises a sequence of FNKPFVFLM (SEQ ID NO: 1), FNKPFVFL[NIe] (SEQ ID NO: 5), FNRPFLVVI (SEQ ID NO: 6), FNRPFLVV[NIe] (SEQ ID NO: 7), FNRPFLMII (SEQ ID NO: 8), or FNRPFLVI[NIe] (SEQ ID NO: 9).

In some aspects, the use of the SERPIN peptide in treating acute or neuropathic pain, nociceptive pain, or inflammatory pain comprises administering a SERPIN peptide comprising an amino acid sequence of X1-Z1-X2-Z2-X3-Z3-F-V-F-L-X4-Z4 (SEQ ID NO: 20), wherein: X1 is V or L; X2 is R or F; X3 is R or K; X4 is M, NIe, or I; Z1 is any amino acid; Z2 is any amino acid; Z3 is any amino acid, and Z4 is a sequence of any five amino acids. In some aspects, the SERPIN peptide comprises an amino acid sequence of X1-Z1-X2-Z2-X3-Z3-F-X4-F-L-Z4-X5 (SEQ ID NO: 21), wherein: X1 is V or L; X2 is F or R; X3 is K or R; X4 is V, L, or M; X5 is a sequence any five amino acids; Z1 is any amino acid; Z2 is a sequence of any two amino acids; Z3 is any amino acid; and Z4 is M, NIe, or I.

In some aspects, the use of the SERPIN peptide in treating acute or neuropathic pain, nociceptive pain, or inflammatory pain comprises administering a SERPIN peptide comprising the sequence of SEQ ID NO: 35. In some aspects, the SERPIN peptide comprises the sequence of SEQ ID NO: 2. In some aspects, the N-terminus of the SERPIN peptide is acetylated. In some aspects, the C-terminus of the SERPIN peptide is amidated. In some aspects, the SERPIN peptide is fused to one or more other peptides to form a fusion peptide or fusion protein. In some aspects, the one or more other peptides is different from the SERPIN peptide. In some aspects, the fusion peptide or fusion protein comprises the SERPIN peptide and an epitope tag, a half-life extender, or both the epitope tag and the half-life extender. In some aspects, the technology includes a pharmaceutical composition comprising the SERPIN peptide and a pharmaceutically effective carrier.

In some embodiments, the use of the SERPIN peptide in treating acute or neuropathic pain, nociceptive pain, or inflammatory pain comprises administering a SERPIN peptide at a therapeutically effective dose or concentration. In some aspects, the pharmaceutical compound is administered at a therapeutically effective dose. In some aspects, the SERPIN peptide is administered at a clinically effective dose or concentration. In some aspects, the pharmaceutical compound is administered at a clinically effective dose. In some aspects, the SERPIN peptide is administered at a dose of between 0.001 mg/kg and 5 mg/kg. In some aspects, the subject is a human. In certain embodiments, the administration is by oral administration, parenteral administration, intradermal administration, transdermal administration, topical administration, or intranasal administration. In some aspects, the composition is administered as a single dose.

In some aspects, the use of the SERPIN peptide in treating acute or neuropathic pain, nociceptive pain, or inflammatory pain comprises administering a SERPIN peptide, wherein the disease or condition is an acute or neuropathic pain. In some aspects, the disease or condition is nociceptive pain. In some aspects, the disease or condition is inflammatory pain. In some aspects, administering the SERPIN peptide reduces pain. In some aspects, administering the SERPIN peptide prevents or reduces the development of pain. In some aspects, administering the SERPIN peptide increases neuronal survival and neurite sprouting.

Uses for SERPIN Peptide in Treating a Disease or Condition Caused by A. alternata

In some embodiments, the present technology includes use of a SERPIN peptide in treating a subject having a disease or condition associated with LRP1 or TSLP, where the disease or condition is caused by A. alternata. In some aspects, the technology includes use of any SERPIN peptide described in this disclosure.

In some aspects, the use of the SERPIN peptide in treating a disease or condition caused by A. alternata comprises administering a SERPIN peptide comprising an amino acid sequence selected from the group consisting of VKFNKPFVFL(NIe)IEQNTK (SEQ ID NO: 35), VKFNKPFVFLMIEQNTK (SEQ ID NO: 2), VKFNKPFVFLM (SEQ ID NO: 25), LRFNRPFLVVI (SEQ ID NO: 29), VRFNRPFLMII (SEQ ID NO: 31), VKFNKPFVFL(NIe) (SEQ ID NO: 40), RFNRPFLVVIR (SEQ ID NO: 41), RFNRPFLMIIR (SEQ ID NO: 42), RFNKPFVFL(NIe)R (SEQ ID NO: 43), RRRFLVVIRRR (SEQ ID NO: 44), RRRFLMIIRRR (SEQ ID NO: 45), RRRFVFL(NIe)RRR (SEQ ID NO: 46), FVFLM (SEQ ID NO: 3), and FVFL(NIe) (SEQ ID NO: 10), wherein the SERPIN peptide is administered to the subject to treat the disease or condition associated with LRP1 or TSLP, wherein the disease or condition is caused by A. alternata.

In some aspects, the use of the SERPIN peptide in treating a disease or condition caused by A. alternata comprises administering a SERPIN peptide comprising the SP16 peptide (SEQ ID NO: 2) or the SP163M peptide (SEQ ID NO: 35). In some aspects, the SERPIN peptide comprises an amino acid sequence that shares at least 95% identity with the SP16 peptide (SEQ ID NO: 2). In some aspects, the SERPIN peptide comprises an amino acid sequence that shares at least 95% identity with the SP163M peptide (SEQ ID NO: 35). In some aspects, the SERPIN peptide comprises an amino acid sequence that shares at least 90% identity with the SP16 peptide (SEQ ID NO: 2). In some aspects, the SERPIN peptide comprises an amino acid sequence that shares at least 90% identity with the SP163M peptide (SEQ ID NO: 35).

In some aspects, the use of the SERPIN peptide in treating a disease or condition caused by A. alternata comprises administering a SERPIN peptide comprising an amino acid sequence of X1-N-X2-P-F-X3-X4-X5-X6, wherein X1 is R or F, X2 is K or R, X3 is V or L, X4 is F, V, or M, X5 is L, V, or I, and X6 is M, I, or NIe. In some aspects, the SERPIN peptide comprises a sequence of FNKPFVFLM (SEQ ID NO: 1), FNKPFVFL[NIe](SEQ ID NO: 5), FNRPFLVVI (SEQ ID NO: 6), FNRPFLVV[NIe] (SEQ ID NO: 7), FNRPFLMII (SEQ ID NO: 8), or FNRPFLVI[NIe] (SEQ ID NO: 9).

In some aspects, the use of the SERPIN peptide in treating a disease or condition caused by A. alternata comprises administering a SERPIN peptide comprising an amino acid sequence of X1-Z1-X2-Z2-X3-Z3-F-V-F-L-X4-Z4 (SEQ ID NO: 20), wherein: X1 is V or L; X2 is R or F; X3 is R or K; X4 is M, NIe, or I; Z1 is any amino acid; Z2 is any amino acid; Z3 is any amino acid; and Z4 is a sequence any five amino acids. In some aspects, the SERPIN peptide comprises an amino acid sequence of X1-Z1-X2-Z2-X3-Z3-F-X4-F-L-Z4-X5 (SEQ ID NO: 21), wherein: X1 is V or L; X2 is F or R; X3 is K or R; X4 is V, L, or M; X5 is a sequence any five amino acids; Z1 is any amino acid; Z2 is a sequence of any two amino acids; Z3 is any amino acid; and Z4 is M, NIe, or I.

In some aspects, the use of the SERPIN peptide in treating a disease or condition caused by A. alternata comprises administering a SERPIN peptide comprising the sequence of SEQ ID NO: 35. In some aspects, the amino acid sequence of the SERPIN peptide comprises the sequence of SEQ ID NO: 2. In some aspects, the N-terminus of the SERPIN peptide is acetylated. In some aspects, the C-terminus of the SERPIN peptide is amidated. In some aspects, the SERPIN peptide is fused to one or more other peptides to form a fusion peptide or fusion protein. In some aspects, the one or more other peptides is different from the SERPIN peptide. In some aspects, the fusion peptide or fusion protein comprises the SERPIN peptide and an epitope tag, a half-life extender, or both the epitope tag and the half-life extender. In some aspects, the technology includes a pharmaceutical composition comprising the SERPIN peptide and a pharmaceutically effective carrier.

In some embodiments, the use of the SERPIN peptide in treating a disease or condition caused by A. alternata comprises administering a SERPIN peptide at a therapeutically effective dose or concentration. In some aspects, the pharmaceutical compound is administered at a therapeutically effective dose. In some aspects, the SERPIN peptide is administered at a clinically effective dose or concentration. In some aspects, the pharmaceutical compound is administered at a clinically effective dose. In some aspects, the SERPIN peptide is administered at a dose of between 0.001 mg/kg and 5 mg/kg. In some aspects, the subject is a human. In some aspects, the administration is by oral administration, parenteral administration, intradermal administration, transdermal administration, topical administration, or intranasal administration. In some aspects, the composition is administered as a single dose.

In some aspects, the use of the SERPIN peptide in treating a disease or condition caused by A. alternata comprises administering a SERPIN peptide, wherein the disease or condition is rhinitis, asthma, dermatitis, or esophageal eosinophilia. In some aspects, the disease or condition is rhinitis. In some aspects, the disease or condition is asthma. In some aspects, the disease or condition is dermatitis. In some aspects, the disease or condition is esophageal eosinophilia. In some aspects, administrating the SERPIN peptide reduces inflammation. In some aspects, administering the SERPIN peptide reduces eosinophilic inflammation.

### REFERENCES

The references, patents, and published patent applications listed below, and all references cited in the specification above, are hereby incorporated by reference in their entirety, as if fully set forth herein.
1. Berta, T., Y. C. Liu, Z. Z. Xu, and R. R. Ji (2013). “Tissue plasminogen activator contributes to morphine tolerance and induces mechanical allodynia via astrocytic IL-1 beta and ERK signaling in the spinal cord of mice.” Neuroscience 247: 376-385.
2. Christia, P., M. Bujak, C. Gonzalez-Quesada, W. Chen, M. Dobaczewski, A. Reddy, and N. G. Frangogiannis (2013). “Systematic characterization of myocardial inflammation, repair, and remodeling in a mouse model of reperfused myocardial infarction.” J Histochem Cytochem 61(8): 555-570.
3. Cooke, J. P. (2019). “Inflammation and Its Role in Regeneration and Repair.” Circ Res 124(8): 1166-1168.
4. Feng, Y., B. Liu, X. Zheng, L. Chen, W. Chen, and Z. Fang (2019). “The protective role of autophagy in sepsis.” Microb Pathoq 131: 106-111.
5. Franchini, M. and M. Montagnana (2011). “Low-density lipoprotein receptor-related protein 1: new functions for an old molecule.” Clin Chem Lab Med 49(6): 967-970.
6. Fregnan, F., L. Muratori, A. R. Simöes, M. G. Giacobini-Robecchi, and S. Raimondo (2012). “Role of inflammatory cytokines in peripheral nerve injury.” Neural reqeneration research 7(29): 2259-2266.
7. Gali, C. C., E. Fanaee-Danesh, M. Zandl-Lang, N. M. Albrecher, C. Tam-Amersdorfer, A. Stracke, V. Sachdev, F. Reichmann, Y. Sun, A. Avdili, M. Reiter, D. Kratky, P. Holzer, A. Lass, K. K. Kandimalla, and U. Panzenboeck (2019). “Amyloid-beta impairs insulin signaling by accelerating autophagy-lysosomal degradation of LRP-1 and IR-β in blood-brain barrier endothelial cells in vitro and in 3XTg-AD mice.” Molecular and Cellular Neuroscience 99: 103390.
8. Gettins, P. G. W. and S. T. Olson (2016). “Inhibitory serpins. New insights into their folding, polymerization, regulation and clearance.” The Biochemical journal 473(15): 2273-2293.
9. Griffin, D., B. Levine, W. Tyor, S. Ubol, and P. Despres (1997). “The role of antibody in recovery from alphavirus encephalitis.” Immunol Rev 159: 155-161.
10. Grosso, R. A., P. V. Subirada Caldarone, M. C. Sanchez, G. A. Chiabrando, M. I. Colombo, and C. M. Fader (2018). “Hemin induces autophagy in a leukemic erythroblast cell line through the LRP1 receptor.” Biosci Rep.
11. Gubler, D. J. (2002). “The global emergence/resurgence of arboviral diseases as public health problems.” Archives of Medical Research 33(4): 330-342.
12. Hashizume, H., J. A. DeLeo, R. W. Colburn, and J. N. Weinstein (2000). “Spinal glial activation and cytokine expression after lumbar root injury in the rat.” Spine (Phila Pa 1976) 25(10): 1206-1217.
13. Hemonnot, A.-L., J. Hua, L. Ulmann, and H. Hirbec (2019). “Microglia in Alzheimer Disease: Well-Known Targets and New Opportunities.” Frontiers in Aging Neuroscience 11(233).
14. Herz, J. and D. K. Strickland (2001). “LRP: a multifunctional scavenger and signaling receptor.” J Clin Invest 108(6): 779-784.
15. Hoffmann, M., H. Kleine-Weber, S. Schroeder, N. Krüger, T. Herrler, S. Erichsen, T. S. Schiergens, G. Herrler, N.-H. Wu, A. Nitsche, M. A. Müller, C. Drosten, and S. Pöhlmann (2020). “SARS-CoV-2 Cell Entry Depends on ACE2 and TMPRSS2 and Is Blocked by a Clinically Proven Protease Inhibitor.” Cell.
16. Hoffmann, M., H. Kleine-Weber, S. Schroeder, N. Krüger, T. Herrler, S. Erichsen, T. S. Schiergens, G. Herrler, N. H. Wu, A. Nitsche, M. A. Müller, C. Drosten, and S. Pöhlmann (2020). “SARS-CoV-2 Cell Entry Depends on ACE2 and TMPRSS2 and Is Blocked by a Clinically Proven Protease Inhibitor.” Cell 181(2): 271-280.e278.
17. Huang, C., Y. Wang, X. Li, L. Ren, J. Zhao, Y. Hu, L. Zhang, G. Fan, J. Xu, X. Gu, Z. Cheng, T. Yu, J. Xia, Y. Wei, W. Wu, X. Xie, W. Yin, H. Li, M. Liu, Y. Xiao, H. Gao, L. Guo, J. Xie, G. Wang, R. Jiang, Z. Gao, Q. Jin, J. Wang, and B. Cao (2020). “Clinical features of patients infected with 2019 novel coronavirus in Wuhan, China.” The Lancet 395(10223): 497-506.
18. Joslin, G., R. J. Fallon, J. Bullock, S. P. Adams, and D. H. Perlmutter (1991). “The SEC receptor recognizes a pentapeptide neodomain of alpha 1-antitrypsin-protease complexes.” J Biol Chem 266(17): 11282-11288.
19. Kawamura, A., D. Baitsch, R. Telgmann, R. Feuerborn, G. Weissen-Plenz, C. Hagedorn, K. Saku, S. M. Brand-Herrmann, A. von Eckardstein, G. Assmann, and J. R. Nofer (2007). “Apolipoprotein E interrupts interleukin-1 beta signaling in vascular smooth muscle cells.” Arterioscler Thromb Vasc Biol 27(7): 1610-1617.
20. Kehn-Hall, K., A. Narayanan, L. Lundberg, G. Sampey, C. Pinkham, I. Guendel, R. Van Duyne, S. Senina, K. L. Schultz, E. Stavale, M. J. Aman, C. Bailey, and F. Kashanchi (2012). “Modulation of GSK-3beta activity in Venezuelan equine encephalitis virus infection.” PLoS One 7(4): e34761.
21. Landowski, L. M., M. Pavez, L. S. Brown, R. Gasperini, B. V. Taylor, A. K. West, and L. Foa (2016). “Low-density Lipoprotein Receptor-related Proteins in a Novel Mechanism of Axon Guidance and Peripheral Nerve Regeneration.” The Journal of biological chemistry 291(3): 1092-1102.
22. Lillis, A. P., L. B. Van Duyn, J. E. Murphy-Ullrich, and D. K. Strickland (2008). “LDL receptor-related protein 1: unique tissue-specific functions revealed by selective gene knockout studies.” Physiol Rev 88(3): 887-918.
23. Mantuano, E., M. S. Lam, M. Shibayama, W. M. Campana, and S. L. Gonias (2015). “The NMDA receptor functions independently and as an LRP1 co-receptor to promote Schwann cell survival and migration.” Journal of Cell Science 128(18): 3478-3488.
24. Matsuyama, S., N. Nao, K. Shirato, M. Kawase, S. Saito, I. Takayama, N. Nagata, T. Sekizuka, H. Katoh, F. Kato, M. Sakata, M. Tahara, S. Kutsuna, N. Ohmagari, M. Kuroda, T. Suzuki, T. Kageyama, and M. Takeda (2020). “Enhanced isolation of SARS-CoV-2 by TMPRSS2-expressing cells.” Proceedings of the National Academy of Sciences 117(13): 7001.
25. May, P. (2013). “The low-density lipoprotein receptor-related protein 1 in inflammation.” Curr Opin Lipidol 24(2): 134-137.
26. May, P., H. H. Bock, and J. R. Nofer (2013). “Low density receptor-related protein 1 (LRP1) promotes anti-inflammatory phenotype in murine macrophages.” Cell Tissue Res 354(3): 887-889.
27. Mehta, P., D. F. McAuley, M. Brown, E. Sanchez, R. S. Tattersall, and J. J. Manson (2020). “COVID-19: consider cytokine storm syndromes and immunosuppression.” The Lancet 395(10229): 1033-1034.
28. Meyer, M. and I. Jaspers (2015). “Respiratory protease/antiprotease balance determines susceptibility to viral infection and can be modified by nutritional antioxidants.” American journal of physiology. Lung cellular and molecular physiology 308(12): L1189-L1201.
29. Mishra, A., X. Yao, A. Saxena, E. M. Gordon, M. Kaler, R. A. Cuento, A. V. Barochia, P. K. Dagur, J. P. McCoy, K. J. Keeran, K. R. Jeffries, X. Qu, Z. X. Yu, and S. J. Levine (2018). “Low-density lipoprotein receptor& #x2013; related protein 1 attenuates house dust mite& #x2013; induced eosinophilic airway inflammation by suppressing dendritic cell& #x2013; mediated adaptive immune responses.” Journal of Allergy and Clinical Immunology 142(4): 1066-1079.e1066.
30. Muehlenbein, M. P., F. B. Cogswell, M. A. James, J. Koterski, and G. V. Ludwig (2006). “Testosterone correlates with Venezuelan equine encephalitis virus infection in macaques.” Virol J 3: 19.
31. Olival, K. J. and P. Daszak (2005). “The ecology of emerging neurotropic viruses.” Journal of Neuroviroloqy 11(5): 441-446.
32. Rauch, J. N., G. Luna, E. Guzman, M. Audouard, C. Challis, Y. E. Sibih, C. Leshuk, I. Hernandez, S. Wegmann, B. T. Hyman, V. Gradinaru, M. Kampmann, and K. S. Kosik (2020). “LRP1 is a master regulator of tau uptake and spread.” Nature 580(7803): 381-385.
33. Ronca, S. E., K. T. Dineley, and S. Paessler (2016). “Neurological Sequelae Resulting from Encephalitic Alphavirus Infection.” Front Microbiol 7: 959.
34. Schoneboom, B. A., K. M. Catlin, A. M. Marty, and F. B. Grieder (2000). “Inflammation is a component of neurodegeneration in response to Venezuelan equine encephalitis virus infection in mice.” J Neuroimmunol 109(2): 132-146.
35. Schoneboom, B. A., J. S. Lee, and F. B. Grieder (2000). “Early expression of IFN-alpha/beta and iNOS in the brains of Venezuelan equine encephalitis virus-infected mice.” J Interferon Cytokine Res 20(2): 205-215.
36. Sharma, A., B. Bhattacharya, R. K. Puri, and R. K. Maheshwari (2008). “Venezuelan equine encephalitis virus infection causes modulation of inflammatory and immune response genes in mouse brain.” BMC Genomics 9: 289.
37. Sharma, A., M. Bhomia, S. P. Honnold, and R. K. Maheshwari (2011). “Role of adhesion molecules and inflammation in Venezuelan equine encephalitis virus infected mouse brain.” Viroloqy Journal 8: 197-197.
38. Shi, Y., E. Mantuano, G. Inoue, W. M. Campana, and S. L. Gonias (2009). “Ligand binding to LRP1 transactivates Trk receptors by a Src family kinase-dependent pathway.” Sci Signal 2(68): ra18.
39. Shi, Y., T. Yamauchi, A. Gaultier, S. Takimoto, W. M. Campana, and S. L. Gonias (2011). “Regulation of cytokine expression by Schwann cells in response to alpha2-macroglobulin binding to LRP1.” J Neurosci Res 89(4): 544-551.
40. Shiga, Y., A. Shiga, P. Mesci, H. Kwon, C. Brifault, J. H. Kim, J. J. Jeziorski, C. Nasamran, S. Ohtori, A. R. Muotri, S. L. Gonias, and W. M. Campana (2019). “Tissue-type plasminogen activator-primed human iPSC-derived neural progenitor cells promote motor recovery after severe spinal cord injury.” Scientific Reports 9(1): 19291.
41. Simon, D., B. Page, M. Vogel, C. Bussmann, C. Blanchard, A. Straumann, and H.-U. Simon (2018). “Evidence of an abnormal epithelial barrier in active, untreated and corticosteroid-treated eosinophilic esophagitis.” Allergy 73(1): 239-247.
42. Steele, K. E. and N. A. Twenhafel (2010). “REVIEW PAPER: pathology of animal models of alphavirus encephalitis.” Vet Pathol 47(5): 790-805.
43. Strickland, D. K., S. C. Muratoglu, and T. M. Antalis (2011). “Serpin-Enzyme Receptors LDL Receptor-Related Protein 1.” Methods Enzymol 499: 17-31.
44. Subramaniyam, D., P. Glader, K. von Wachenfeldt, J. Burneckiene, T. Stevens, and S. Janciauskiene (2006). “C-36 peptide, a degradation product of alpha1-antitrypsin, modulates human monocyte activation through LPS signaling pathways.” Int J Biochem Cell Biol 38(4): 563-575.
45. Taylor, K. G. and S. Paessler (2013). “Pathogenesis of Venezuelan equine encephalitis.” Vet Microbiol 167(1-2): 145-150.
46. Toldo, S., D. Austin, A. G. Mauro, E. Mezzaroma, B. W. Van Tassell, C. Marchetti, S. Carbone, S. Mogelsvang, C. Gelber, and A. Abbate (2017). “Low-Density Lipoprotein Receptor-Related Protein-1 Is a Therapeutic Target in Acute Myocardial Infarction.” JACC Basic Transl Sci 2(5): 561-574.
47. Wang, Y., K. Jiang, Q. Zhang, S. Meng, and C. Ding (2018). “Autophagy in Negative-Strand RNA Virus Infection.” Frontiers in Microbiology 9(206).
48. Weaver, S. C., C. Ferro, R. Barrera, J. Boshell, and J. C. Navarro (2004). “Venezuelan equine encephalitis.” Annu Rev Entomol 49: 141-174.
49. Wujak, L., P. Markart and M. Wygrecka (2016). “The low density lipoprotein receptor-related protein (LRP) 1 and its function in lung diseases.” Histol Histopathol 31(7): 733-745.
50. Wujak, L., J. Schnieder, L. Schaefer, and M. Wygrecka (2018). “LRP1: A chameleon receptor of lung inflammation and repair.” Matrix Biology 68-69: 366-381.
51. Yang, L., C.-C. Liu, H. Zheng, T. Kanekiyo, Y. Atagi, L. Jia, D. Wang, A. N'songo, D. Can, H. Xu, X.-F. Chen, and G. Bu (2016). “LRP1 modulates the microglial immune response via regulation of JNK and NF-κB signaling pathways.” Journal of Neuroinflammation 13(1): 304.
52. Yang, X., Y. Yu, J. Xu, H. Shu, J. a. Xia, H. Liu, Y. Wu, L. Zhang, Z. Yu, M. Fang, T. Yu, Y. Wang, S. Pan, X. Zou, S. Yuan, and Y. Shang (2020). “Clinical course and outcomes of critically ill patients with SARS-CoV-2 pneumonia in Wuhan, China: a single-centered, retrospective, observational study.” The Lancet Respiratory Medicine.
53. Yoon, C., E. A. Van Niekerk, K. Henry, T. Ishikawa, S. Orita, M. H. Tuszynski, and W. M. Campana (2013). “Low-density Lipoprotein Receptor-related Protein 1 (LRP1)-dependent Cell Signaling Promotes Axonal Regeneration.” Journal of Biological Chemistry 288(37): 26557-26568.
54. Zlokovic, B. V., R. Deane, A. P. Sagare, R. D. Bell, and E. A. Winkler (2010). “Low-density lipoprotein receptor-related protein-1: a serial clearance homeostatic mechanism controlling Alzheimer's amyloid P-peptide elimination from the brain.” Journal of neurochemistry 115(5): 1077-1089.
55. Zurhove, K., C. Nakajima, J. Herz, H. H. Bock, and P. May (2008). “Gamma-secretase limits the inflammatory response through the processing of LRP1.” Science signaling 1(47): ra15-ra15.
56. Sadri S., N. Hirosawa, J. Le, et al. “Tumor necrosis factor receptor-1 is selectively sequestered into Schwann cell extracellular vesicles where it functions as TNFα decoy.” Glia. 2021. Online ahead of print. doi:10.1002/glia.24098.
57. Orita S., K. Henry, E. Mantuano, et al. “Schwann cell LRP1 regulates Remak bundle ultrastructure and axonal interactions to prevent neuropathic pain.” J Neurosci. 2013; 33(13):5590-5602. doi:10.1523/jneur osci.3342-12.2013.
58. Garcia-Fernandez P., N. Uceyler, and C. Sommer. “From the low-density lipoprotein receptor related protein 1 to neuropathic pain: a potentially novel target.” Pain. 2021; 6(1):e898. doi:10.1097/PR9.00000 00000 000898.
59. Seltzer Z., R. Dubner, and Y. Shir. “A novel behavioral model of neuropathic pain disorders produced in rats by partial sciatic nerve injury.” Pain. 1990; 43(2):205-218. doi:10.1016/0304-3959(90)91074-S.
60. Chaplan S. R., F. W. Bach, J. W. Pogrel, J. M. Chung, and T. L. Yaksh. “Quantitative assessment of tactile allodynia in the rat paw.” J Neurosci Methods. 1994; 53(1):55-63. doi:10.1016/0165-0270(94)90144-9.
61. Poplawski G., T. Ishikawa, C. Brifault, et al. “Schwann cells regulate sensory neuron gene expression before and after peripheral nerve injury.” Glia. 2018; 66(8):1577-1590. doi:10.1002/glia.23325.
62. Hunskaar S. and K. Hole. “The formalin test in mice: dissociation between inflammatory and non-inflammatory pain.” Pain. 1987; 30(1):103-114. doi:10.1016/0304-3959(87)90088-1.
63. Kohro Y., T. Matsuda, K. Yoshihara, et al. “Spinal astrocytes in superficial laminae gate brainstem descending control of mechanosensory hypersensitivity.” Nat Neurosci. 2020; 23:1376-1387. doi:10.1038/s4159 3-020-00713-4.
64. Holzer P. “Capsaicin: cellular targets, mechanisms of action, and selectivity for thin sensory neurons.” Pharmacol Rev. 1991; 43(2):143-201.
65. Caterina M. J., A. Leffler, A. B. Malmberg, et al. “Impaired nociception and pain sensation in mice lacking the capsaicin receptor.” Science. 2000; 288(5464):306-313. doi:10.1126/science.288.5464.306.

1. A method of reducing inflammation in a subject having a disease or condition associated with LRP1 or TSLP, comprising administering a SERPIN peptide comprising an amino acid sequence selected from the group consisting of VKFNKPFVFLMIEQNTK (SEQ ID NO: 2), VKFNKPFVFLM (SEQ ID NO: 25), LRFNRPFLVVI (SEQ ID NO: 29), VRFNRPFLMII (SEQ ID NO: 31), VKFNKPFVFL(NIe) (SEQ ID NO: 40), RFNRPFLVVIR (SEQ ID NO: 41), RFNRPFLMIIR (SEQ ID NO: 42), RFNKPFVFL(NIe)R (SEQ ID NO: 43), RRRFLVVIRRR (SEQ ID NO: 44), RRRFLMIIRRR (SEQ ID NO: 45), RRRFVFL(NIe)RRR (SEQ ID NO: 46), FVFLM (SEQ ID NO: 3), and FVFL(NIe) (SEQ ID NO: 10) to the subject to reduce inflammation associated with the disease or condition associated with LRP1 or TSLP.

2-7. (canceled)

8. The method of claim 1, wherein the SERPIN peptide is administered at a dose of between 0.001 mg/kg and 5 mg/kg.

9. (canceled)

10. The method of claim 1, wherein the administration is by oral administration, parenteral administration, intradermal administration, transdermal administration, topical administration, or intranasal administration.

11. The method of claim 1, wherein the SERPIN peptide is administered as a single dose.

12. The method of claim 1, wherein the disease or condition is caused by A. alternata.

13. The method of claim 1, wherein the disease or condition is rhinitis, asthma, dermatitis, or esophageal eosinophilia.

14-21. (canceled)

22. The method of claim 1, wherein the disease or condition is an eosinophilic driven disease (EDD).

23. The method of claim 1, wherein the disease or condition is eosinophilic esophagitis (EoE), eosinophilic asthma, atopic dermatitis, pruritis, nasal polyps, or chronic spontaneous urticaria.

24-25. (canceled)

26. The method of claim 23, wherein the SERPIN peptide is administered by topical administration.

27. The method of claim 1, wherein the disease or condition is an allergic reaction, allergic inflammation, or eosinophilic driven allergic disease.

28-60. (canceled)

61. A method of treating a subject having a disease or condition associated with LRP1, comprising administering a SERPIN peptide comprising an amino acid sequence of VKFNKPFVFL(NIe)IEQNTK (SEQ ID NO: 35) to the subject to treat the disease or condition associated with LRP1, wherein the disease or condition is acute or neuropathic pain, nociceptive pain, or inflammatory pain.

62-71. (canceled)

72. The method of claim 61, wherein the disease or condition is acute or neuropathic pain.

73. The method of claim 61, wherein the disease or condition is nociceptive pain.

74. The method of claim 61, wherein the disease or condition is inflammatory pain.

75. The method of claim 61, wherein administration of the SERPIN peptide results in reduced pain.

76. The method of claim 61, wherein administration of the SERPIN peptide prevents or reduces the development of pain.

77-92. (canceled)

93. A method of treating a subject having a disease or condition associated with LRP1 or TSLP, comprising administering a SERPIN peptide comprising an amino acid sequence selected from the group consisting of VKFNKPFVFL(NIe)IEQNTK (SEQ ID NO: 35) to the subject to treat the disease or condition associated with LRP1 or TSLP, wherein the disease or condition is caused by A. alternata.

94-103. (canceled)

104. The method of claim 93, wherein the disease or condition is rhinitis, asthma, dermatitis, or esophageal eosinophilia.

105-108. (canceled)

109. The method of claim 93, wherein administrating the SERPIN peptide reduces inflammation.

110. The method of claim 93, wherein administering the SERPIN peptide reduces eosinophilic inflammation.

111-128. (canceled)


SEQUENCE LISTING (46 sequences)

SEQ ID NO: 1 - protein, 9 aa, Homo sapiens
FNKPFVFLM

SEQ ID NO: 2 - protein, 17 aa, Homo sapiens
VKFNKPFVFLMIEQNTK

SEQ ID NO: 3 - protein, 5 aa, Homo sapiens
FVFLM

SEQ ID NO: 4 - skipped

SEQ ID NO: 5 - protein, 9 aa, synthetic construct
  MOD_RES 9: note = Nle
FNKPFVFLX

SEQ ID NO: 6 - protein, 9 aa, synthetic construct
FNRPFLVVI

SEQ ID NO: 7 - protein, 9 aa, synthetic construct
  MOD_RES 9: note = Nle
FNRPFLVVX

SEQ ID NO: 8 - protein, 9 aa, synthetic construct
  MOD_RES 9: note = Nle
FNRPFLMII

SEQ ID NO: 9 - protein, 9 aa, synthetic construct
FNRPFLVIX

SEQ ID NO: 10 - protein, 5 aa, synthetic construct
  MOD_RES 5: note = Nle
FVFLX

SEQ ID NO: 11 - protein, 5 aa, synthetic construct
FLVVI

SEQ ID NO: 12 - protein, 5 aa, synthetic construct
  MOD_RES 5: note = Nle
FLVVX

SEQ ID NO: 13 - protein, 5 aa, synthetic construct
FLMII

SEQ ID NO: 14 - protein, 5 aa, synthetic construct
  MOD_RES 5: note = Nle
FLMIX

SEQ ID NO: 15-16 - skipped (2 entries)

SEQ ID NO: 17 - protein, 31 aa, synthetic construct
  VAR_SEQ 22..31: note = each X is one basic amino acid or none
  VAR_SEQ 1..10: note = each X is one basic amino acid or none
XXXXXXXXXXRFNRPFLVVIRXXXXXXXXXX

SEQ ID NO: 18 - protein, 31 aa, synthetic construct
  VAR_SEQ 1..10: note = each X is one basic amino acid or none
  VAR_SEQ 22..31: note = each X is one basic amino acid or none
XXXXXXXXXXRFNRPFLMIIRXXXXXXXXXX

SEQ ID NO: 19 - protein, 31 aa, synthetic construct
  VAR_SEQ 1..10: note = each X is one basic amino acid or none
  VAR_SEQ 22..31: note = each X is one basic amino acid or none
  MOD_RES 20: note = Nle
XXXXXXXXXXKFNKPFVFLXRXXXXXXXXXX

SEQ ID NO: 20 - protein, 16 aa, synthetic construct
  VAR_SEQ 11: note = M, Nle, or I
XXXXXXFVFLXXXXXX

SEQ ID NO: 21 - skipped

SEQ ID NO: 22 - protein, 21 aa, synthetic construct
FPKMVPQFNTELKIFPEVNIK

SEQ ID NO: 23 - protein, 33 aa, Homo sapiens
  REGION 8..16: note = SP16 peptide core sequence including the LRP1 binding site
SIPPEVKFNKPFVFLMIEQNTKSPLFMGKVVNP

SEQ ID NO: 24 - protein, 17 aa, Homo sapiens
  REGION 3..11: note = SP16 peptide core sequence including the LRP1 binding site
VKFNKPFVFLMIEQNTK

SEQ ID NO: 25 - protein, 11 aa, Homo sapiens
  REGION 3..11: note = SP16 peptide core sequence including the LRP1 binding site
VKFNKPFVFLM

SEQ ID NO: 26 - protein, 23 aa, synthetic construct
  REGION 6..14: note = SP16 peptide core sequence including the LRP1 binding site
RRRVKFNKPFVFLMIEQNTKRRR

SEQ ID NO: 27 - protein, 17 aa, synthetic construct
  REGION 6..14: note = SP16 peptide core sequence including the LRP1 binding site
RRRVKFNKPFVFLMRRR

SEQ ID NO: 28 - protein, 17 aa, synthetic construct
LRFNRPFLVVIFSTSTQ

SEQ ID NO: 29 - protein, 11 aa, synthetic construct
LRFNRPFLVVI

SEQ ID NO: 30 - protein, 17 aa, synthetic construct
RRRLRFNRPFLVVIRRR

SEQ ID NO: 31 - protein, 11 aa, synthetic construct
VRFNRPFLMII

SEQ ID NO: 32 - protein, 17 aa, synthetic construct
RRRVRFNRPFLMIIRRR

SEQ ID NO: 33 - protein, 8 aa, synthetic construct
VRFNRPFL

SEQ ID NO: 34 - protein, 14 aa, synthetic construct
RRRVRFNRPFLRRR

SEQ ID NO: 35 - protein, 17 aa, synthetic construct
  MOD_RES 11: note = Nle
VKFNKPFVFLXIEQNTK

SEQ ID NO: 36-39 - skipped (4 entries)

SEQ ID NO: 40 - protein, 11 aa, synthetic construct
  MOD_RES 11: note = Nle
VKFNKPFVFLX

SEQ ID NO: 41 - protein, 11 aa, synthetic construct
RFNRPFLVVIR

SEQ ID NO: 42 - protein, 11 aa, synthetic construct
RFNRPFLMIIR

SEQ ID NO: 43 - protein, 11 aa, synthetic construct
  MOD_RES 10: note = Nle
RFNKPFVFLXR

SEQ ID NO: 44 - protein, 11 aa, synthetic construct
RRRFLVVIRRR

SEQ ID NO: 45 - protein, 11 aa, synthetic construct
RRRFLMIIRRR

SEQ ID NO: 46 - protein, 11 aa, synthetic construct
  MOD_RES 8: note = Nle
RRRFVFLXRRR